(19) United States
(12) Patent Application Publication
(10) Pub. No.: US 2025/0344813 A1
(43) Pub. Date: Nov. 13, 2025

(54) METHOD FOR FORMING INTEGRAL FASTENERS IN A SUBSTRATE

(71) Applicant: The Procter & Gamble Company, Cincinnati, OH (US)

(72) Inventors: Todd Douglas LENSER, Liberty Township, OH (US); Uwe SCHNEIDER, Cincinnati, OH (US); Christine Ann METHENA, Cincinnati, OH (US); Randall Allen MYERS, Fairfield Township, OH (US)

(21) Appl. No.: 19/202,314

(22) Filed: May 8, 2025

Related U.S. Application Data
(60) Provisional application No. 63/645,177, filed on May 10, 2024.

(51) Int. Cl. A44B 18/00; B29C 51/22; B29C 51/42; B29L 31/00

(52) U.S. Cl. CPC A44B 18/0049; A44B 18/0061; B29C 51/22; B29C 51/421; B29L 2031/729

(57) ABSTRACT

A method of creating projections in a substrate is presented. A first device is provided with an outer surface defining recesses, where each recess has a first portion proximate the outer surface with a first perimeter and first depth and a second portion distal the outer surface with a second perimeter and second depth. The first perimeter is larger than the second perimeter and the first depth is less than the second depth. The first portion of the recesses substantially surrounds the second portion of the recesses. The method also includes forming a nip between a source of vibration energy and the outer surface and conveying the substrate through the nip to create precursor projections. The method also includes heating portions of the precursor projections and applying pressure to the heated portions to flatten the portions and increase a perimeter of the portions to form the projections.

### CROSS REFERENCE TO RELATED APPLICATION

This application claims the benefit, under 35 U.S.C. § 119(e), of U.S. Provisional Patent Application No. 63/645,177, filed on May 10, 2024, the entire disclosure of which is incorporated herein by reference in its entirety.

### FIELD

The present disclosure relates generally to mechanical fasteners, such as hook and loop fasteners or touch fasteners and more specifically to a method for producing hook fasteners using vibration energy.

### BACKGROUND

Conventional methods are known for forming fasteners in a substrate using vibration energy, such as ultrasonic energy. In this conventional method, a non-woven film is passed through a nip between a molding roll having recesses and a source of vibration energy, such as a sonotrode. As the substrate moves through the nip, some of the substrate (e.g. polypropylene) is displaced into the recesses of the molding roll to form hooks in the film or sheet that emerges from the nip. In some conventional methods, J-shaped hooks are formed based on J-shaped recesses in the molding roll.

Another conventional method for forming fasteners in a substrate starts with a substrate having cylinder shaped precursor projections thereon. Tips of these cylinder shaped precursor projections are first heated after which a first roller impacts the heated tip to form a T-shaped pin projection. A second roller then impacts the outer perimeter of the T-shaped projection to form an arrow-shaped projection.

The discussion of shortcomings and needs existing in the field prior to the present disclosure is in no way an admission that such shortcomings and needs were recognized by those skilled in the art prior to the present disclosure.

### SUMMARY

The inventors of the present invention recognized various drawbacks of the conventional methods for integrally forming fasteners in a substrate using vibration energy (e.g. ultrasonic energy). The inventors noted that although these conventional methods effectively generate fasteners, such as J-hooks, in the substrate using vibration energy, they do so at a relatively slow rate (e.g. about 40-45 meters per minute). The inventors realized that if the method could be improved to generate fasteners in the substrate at much higher rate (e.g. about 300 meters per minute), this would substantially increase the overall economic efficiency of the method. The inventors achieved this improvement by modifying the shape of the recesses in the molding roll so that a greater percentage of an average substrate area for each recess is conveyed into each recess to form the fasteners. This in turn increases the maximum speed at which the substrate can be moved through the nip and thus consequently increases the overall efficiency of the method.

The inventors also recognized that rather than generating J-shaped hooks as in conventional methods, the method disclosed herein could be designed to generate omnidirectional hooks (e.g. mushroom shaped hooks) at a much greater density (e.g. 2000 hooks per square inch) than the conventionally generated J-shaped hooks (e.g. 600 hooks per square inch).

Various embodiments solve the above-mentioned problems and provide methods and devices useful for integrally forming omnidirectional fasteners in a substrate at a greater efficiency than conventional methods.

In a first set of embodiments, a method of creating projections in a substrate is presented. The method includes providing a first device comprising an outer surface. The outer surface defines a plurality of recesses. The recesses include a first portion proximate to the outer surface and having a first perimeter and a first depth. The recesses also include a second portion distal from the outer surface and having a second perimeter and a second depth. The first perimeter is larger than the second perimeter, and the first depth is less than the second depth. The first portion of the recesses substantially surrounds the second portion of the recesses. The method also includes providing a second device comprising a source of vibration energy. The method also includes forming a nip between the source of vibration energy and the outer surface. The method also includes conveying the substrate through the nip in a machine direction to create a plurality of precursor projections from a portion of the substrate. The precursor projections have a first portion having a first perimeter and a first depth corresponding to the first perimeter and the first depth of the first portion of the recesses. The precursor projections have a second portion having a second perimeter and a second depth corresponding to the second perimeter and the second depth of the second portion of the recesses. The first portion of the precursor projections substantially surrounds the second portion of the precursor projections. The plurality of precursor projections comprise proximal regions proximate to a plane of the substrate and distal regions distal from the plane of the substrate. The method also includes heating portions of the distal regions to a temperature at or above a melting temperature of the substrate. The method also includes applying pressure to the portions of the distal regions to flatten the portions of the distal regions and increase a perimeter of the portion of the distal regions to form the projections.

In a second set of embodiments, a method of creating projections in a substrate is presented which is similar to the first set of embodiments with the exception that the second device is a source of heat or pressure.

These and other features, aspects, and advantages of various embodiments will become better understood with reference to the following description, figures, and claims.

### BRIEF DESCRIPTION OF THE DRAWINGS

Many aspects of this disclosure can be better understood with reference to the following figures, which illustrate examples according to various embodiments.

FIG. 1 is an example according to various embodiments illustrating a schematic view of the apparatus that is used to integrally form precursor projections in a substrate;

FIG. 2A is an example according to various embodiments illustrating a cross-sectional side view of a recess formed in an outer surface of the molding roll in the apparatus of FIG. 1;

FIG. 2B is an example according to various embodiments illustrating a top view of the recess formed in the outer surface of the molding roll in the apparatus of FIG. 1;

FIG. 3A is an example according to various embodiments illustrating a cross-sectional side view of a precursor projection formed along the film or sheet emerging from the nip in the apparatus of FIG. 1;

FIG. 3B is an example according to various embodiments illustrating an end view of the precursor projection formed along the film or sheet emerging from the nip in the apparatus of FIG. 1;

FIG. 3C is an example according to various embodiments illustrating a side view of the precursor projection formed along the film or sheet emerging from the nip in the apparatus of FIG. 1;

FIG. 4A is an example according to various embodiments illustrating a top view of the outer surface of the molding roll in the apparatus of FIG. 1;

FIG. 4B is an example according to various embodiments illustrating a cross-sectional view of the molding roll in FIG. 4A taken along the line 4B-4B;

FIG. 4C is an example according to various embodiments illustrating a top view of the outer surface of the molding roll with offset recesses in the apparatus of FIG. 1;

FIG. 4D is an example according to various embodiments illustrating a cross-sectional view of the molding roll in FIG. 4C taken along the line 4D-4D;

FIG. 5A illustrates a top view of the outer surface of the molding roll in a conventional apparatus for integrally forming projections in a substrate;

FIG. 5B illustrates a cross-sectional view of the molding roll in FIG. 5A taken along the line 5B-5B;

FIG. 5C illustrates a top view of the outer surface of the molding roll with offset recesses in a conventional apparatus for integrally forming projections in a substrate;

FIG. 5D illustrates a cross-sectional view of the molding roll in FIG. 5C taken along the line 5D-5D;

FIG. 6A is an example according to various embodiments illustrating a schematic view of an apparatus to integrally form projections in a substrate based on an input of the precursor projections formed in FIG. 1;

FIG. 6B is an example according to various embodiments illustrating a schematic view of the precursor projections, the projections having a flattened distal region and the projections having a mushroom shape at different stages of the apparatus of FIG. 6A;

FIG. 6C is an example according to various embodiments illustrating a schematic view of the apparatus of FIG. 6A where one of the components is moved from a first position to a second position to bypass a heated device;

FIG. 7A is an example according to various embodiments illustrating a side view of the precursor projection with a flattened distal region based on impact with the anvil of FIG. 6A;

FIG. 7B is an example according to various embodiments illustrating a side perspective view of the projection with a mushroom shape based on the flattened distal region being rounded towards the substrate using the deformable device of FIG. 6A;

FIG. 7C is an example according to various embodiments illustrating a side perspective view of the projection with a mushroom shape generated by the apparatus of FIG. 6A with one or more dimensional parameters;

FIG. 7D is an example according to various embodiments illustrating a top view of a shaped patch of formed projections in the substrate using the apparatus depicted in FIGS. 1 and 6A;

FIGS. 7E and 7F is an example of a conventional planar J-shaped hook being formed by a conventional molding roll having planar J-shaped recesses, and

FIG. 8 is an example according to various embodiments illustrating a flowchart depicting a method for integrally forming projections in a substrate.

FIGS. 9A-9C illustrate data related to projections.

FIGS. 10A-10C illustrate data related to projections.

It should be understood that the various embodiments are not limited to the examples illustrated in the figures.

### DETAILED DESCRIPTION

### Introduction and Definitions

This disclosure is written to describe the invention to a person having ordinary skill in the art, who will understand that this disclosure is not limited to the specific examples or embodiments described. The examples and embodiments are single instances of the invention which will make a much larger scope apparent to the person having ordinary skill in the art. Unless defined otherwise, all technical and scientific terms used herein have the same meaning as commonly understood by the person having ordinary skill in the art. It is also to be understood that the terminology used herein is for the purpose of describing examples and embodiments only, and is not intended to be limiting, since the scope of the present disclosure will be limited only by the appended claims.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and embodiments described herein are for illustrative purposes only and that various modifications or changes in light thereof will be suggested to the person having ordinary skill in the art and are to be included within the spirit and purview of this application. Many variations and modifications may be made to the embodiments of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All numeric values are herein assumed to be modified by the term “about,” whether or not explicitly indicated. The term “about” generally refers to a range of numbers that one of skill in the art would consider equivalent to the recited value (for example, having the same function or result). In many instances, the term “about” may include numbers that are rounded to the nearest significant figure.

In everyday usage, indefinite articles (like “a” or “an”) precede countable nouns and noncountable nouns almost never take indefinite articles. It must be noted, therefore, that, as used in this specification and in the claims that follow, the singular forms “a,” “an,” and “the” include plural referents unless the context clearly dictates otherwise. Thus, for example, reference to “a support” includes a plurality of supports. Particularly when a single countable noun is listed as an element in a claim, this specification will generally use a phrase such as “a single.” For example, “a single support.”

Unless otherwise specified, all percentages indicating the amount of a component in a composition represent a percent by weight of the component based on the total weight of the composition. The term “mol percent” or “mole percent” generally refers to the percentage that the moles of a particular component are of the total moles that are in a mixture. The sum of the mole fractions for each component in a solution is equal to 1.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

In this specification and in the claims that follow, reference will be made to a number of terms that shall be defined to have the following meanings unless a contrary intention is apparent.

“Standard temperature and pressure” generally refers to 25° C. and 1 atmosphere. Standard temperature and pressure may also be referred to as “ambient conditions.” Unless indicated otherwise, parts are by weight, temperature is in ° C., and pressure is at or near atmospheric. The terms “elevated temperatures” or “high-temperatures” generally refer to temperatures of at least 100° C.

“Absorbent article” refers to devices that absorb and contain liquid, and more specifically, refers to devices that are placed against or in proximity to the body of the wearer to absorb and to contain various exudates discharged from the body.

“Machine direction” (MD) refers to the direction of material flow through a process. In addition, relative placement and movement of material can be described as flowing in the machine direction through a process from upstream in the process to downstream in the process.

“Cross direction” (CD) refers to a direction that is generally perpendicular to the machine direction.

“Average watershed area” refers to a total area of a projection containing region of an outer surface of a molding roll encompassing a plurality of projections divided by a total number of the plurality of projections.

“Ported inline watershed area” refers to an area along the outer surface of the molding roll having a CD width defined by a diameter of a top portion of a recess at the outer surface of the molding roll and a length defined as a spacing in the MD between adjacent recesses along the outer surface.

“Cavity inline watershed area” refers to an area along the outer surface of the molding roll having a width defined by a diameter of a bottom portion of the recess in the outer surface of the molding roll and a length defined as a spacing in the MD between adjacent recesses along the outer surface.

### Method Overview

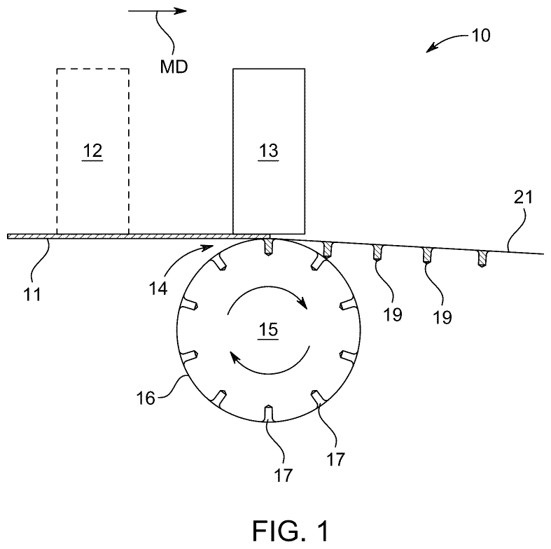

An apparatus will first be discussed that is used to form precursor projections in a substrate. FIG. 1 is an example according to various embodiments illustrating a schematic view of an apparatus 10 that is used to integrally form precursor projections 19 in a substrate 11. The apparatus 10 includes a first device with an outer surface 16. In some embodiments, the first device is a molding roll 15. The outer surface 16 defines a plurality of recesses 17. Although FIG. 1 depicts that the precursor projections 19 are cylindrical, this is for ease of illustration.

As further shown in FIG. 1, the apparatus 10 also includes a second device 13 that is a source of vibration energy (e.g. ultrasonic energy). In one embodiment, the second device 13 is a rotary sonotrode. In another embodiment, the second device 13 is a blade sonotrode. FIG. 1 depicts that a nip 14 is formed between the second device 13 and the outer surface 16 of the molding roll 15. However, in other embodiments the second device 13 is a source or heat or pressure and thus need not be a source of ultrasonic energy, such as a sonotrode.

During operation of the apparatus 10, the substrate 11 is conveyed through the nip 14 in the machine direction (MD) to create a plurality of precursor projections 19 from a portion of the substrate 11. The plurality of precursor projections 19 are integrally formed on a film or sheet 21 that emerges from the nip 14.

In some embodiments, the substrate 11 is a nonwoven or a film. In still other embodiments, the substrate 11 includes one or more layers. The substrate 11 is made from thermoplastic, such as polyolefins, which may include polypropylene, polyethylene, PET, or combinations thereof. The substrate 11 may be, but need not be limited to, a film, sheet, web, nonwoven, composite, laminate, or other form, or may be portions of a film, sheet, web, nonwoven, laminate or substrate thermoplastic material, portions of which may be used as a component of a touch fastener, for instance on an absorbent article. In their use on absorbent article, touch fasteners may be attached to a “side tab” or “ear” that the consumer uses to secure the absorbent article the wearer. These tabs may be constructed with a piece of extensible material to allow the side tab to stretch and flex when attached or when the wearer moves. The touch fasteners may also be used in a two-point fastening system on an absorbent article, where the component is positioned on a landing zone or outer cover of the absorbent article. The present disclosure further contemplates the use of pre-formed film, sheet, web, composite, laminate, etc. as a substrate material.

Although FIG. 1 depicts a plurality of regular spaced precursor projections 19 formed on the film or sheet 21, in other embodiments, one or more patches of the precursor projections 19 are formed intermittently on the film or sheet 21 such that each patch is spaced apart along the film or sheet 21. Although FIG. 1 depicts a single row of precursor projections 19 along the MD with each precursor projections 19 aligned in the cross direction (CD), in other embodiments a plurality of rows of precursor projections 19 are provided along the MD, where each row is spaced apart in the CD.

In some embodiments, the substrate 11 is heated upstream of the nip 14. In these embodiments, the apparatus 10 may include a heated device 12 which heats the substrate 11 upstream of the nip 14. The heated device 12 imparts thermal energy to a portion of the substrate 11 upstream of the nip 14 to heat the portion of the substrate 11 to a temperature below a melting temperature of the portion of the substrate 11. This heating of the substrate 11 upstream of the nip 14 may be performed to soften the thermoplastic, which may reduce said substrate's elastic modulus, yield stress, and/or apparent viscosity.

The operation of the apparatus 10 is similar to what is disclosed in U.S. Pat. No. 10,076,162 B1, with the exception of various features discussed herein (e.g. dimensions or shape of the recesses 17, speed of the substrate 11, etc.). During operation of the apparatus 10, the molding roll 15 is rotated (e.g. in a clockwise direction, as shown in FIG. 1) and the second device 13 is activated such that vibration energy therefrom causes some of the substrate 11 material to enter the recesses 17. As the mold roll 15 continues to rotate, the formed precursor projections 19 emerge from the recesses 17 integrally formed on the film or sheet 21.

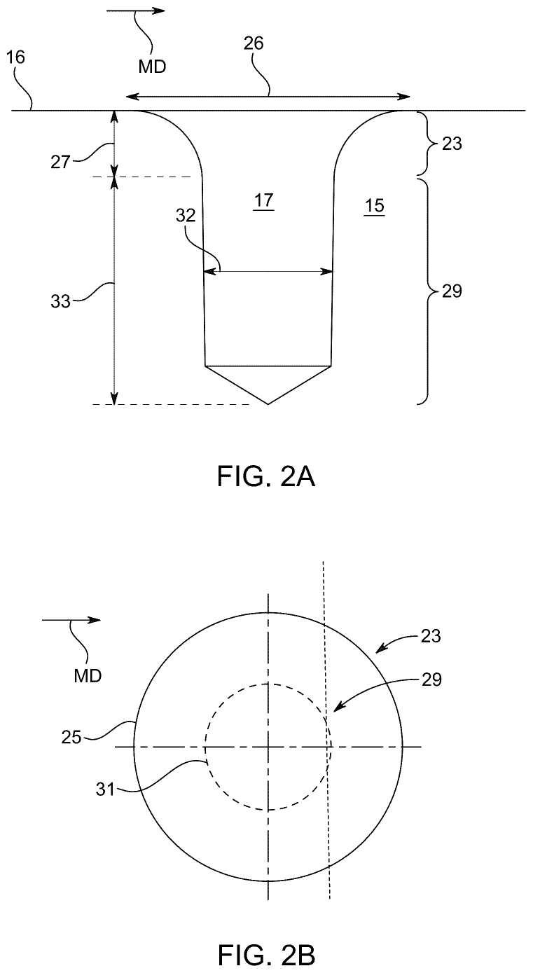

The dimensions and shape of the recesses 17 along the outer surface 16 of the molding roll 15 will now be discussed herein. FIG. 2A is an example according to various embodiments illustrating a cross-sectional side view of the recess 17 formed in the outer surface 16 of the molding roll 15 in the apparatus 10 of FIG. 1. FIG. 2B is an example according to various embodiments illustrating a top view of the recess 17 formed in the outer surface 16 of the molding roll 15 in the apparatus of FIG. 1. In some embodiments, each recess 17 formed in the outer surface 16 has a same shape and/or dimensional parameter values. In other embodiments, multiple recesses 17 formed in the outer surface 16 have a different shape and/or different dimensional parameter values.

As shown in FIGS. 2A and 2B, in one embodiment the recess 17 has a first portion 23 proximate to the outer surface 16 of the molding roll 15. The first portion 23 has a first perimeter 25 (FIG. 2B), a first diameter 26 (FIG. 2A) and a first depth 27 (FIG. 2A). For purposes of this description, “first depth” means a dimension which the first portion 23 extends in a direction perpendicular to the MD (e.g. measured from the outer surface 16). In this embodiment, the first perimeter 25 and the first diameter 26 are measured in a plane that is parallel to the outer surface 16 and the first depth 27 is measured in a direction that is perpendicular to the plane that is parallel to the outer surface 16. In an example embodiment, the first perimeter 25 is about 0.94 mm or in a range from about 0.6 mm to about 1.3 mm; the first diameter 26 is about 0.3 mm or in a range from about 0.2 mm to about 0.4 mm; and the first depth 27 is about 0.08 mm or in a range from about 0.05 mm to about 0.15 mm.

As further shown in FIGS. 2A and 2B, the recess 17 also includes a second portion 29 distal from the outer surface 16 of the molding roll 15. The second portion 29 has a second perimeter 31 (FIG. 2B), a second diameter 32 (FIG. 2A) and a second depth 33 (FIG. 2A). For purposes of this description, “second depth” means a dimension which the second portion 29 extends in a direction perpendicular to the MD and is measured from a boundary between the first and second portions 23, 29 (FIG. 2A). In this embodiment, the second perimeter 31 and the second diameter 32 are measured in the plane that is parallel to the outer surface 16 and the second depth 33 is measured in a direction that is perpendicular to the plane that is parallel to the outer surface 16. In an example embodiment, the second perimeter 31 is about 0.4 mm or in a range from about 0.25 mm to about 1.0 mm; the second diameter 32 is about 0.14 mm or in a range from about 0.05 mm to about 0.3 mm; and the second depth 33 is about 0.27 mm or in a range from about 0.15 mm to about 0.5 mm. In one example embodiment, the first perimeter 25 is larger than the second perimeter 31 and the first diameter 26 is larger than the second diameter 32. In another example embodiment, the first depth 27 is less than the second depth 33. As depicted in FIG. 2B, the first portion 23 of the recess 17 substantially surrounds the second portion 29 of the recess 17. In another example embodiment, a radius of curvature of the recess 17 in the first portion 23 is about 0.08 or in a range from about 0.020 to about 0.150.

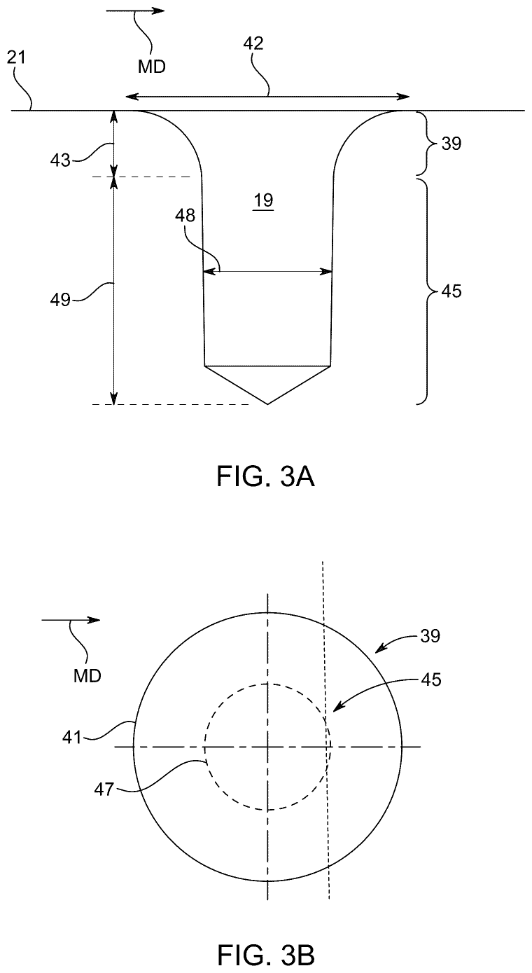

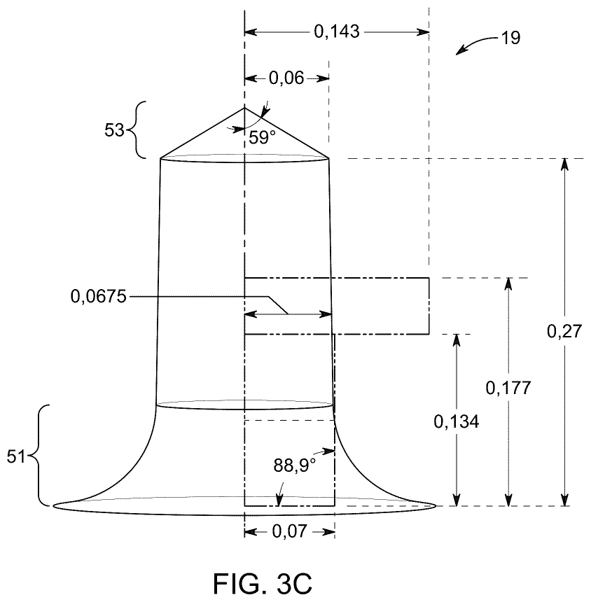

The dimensions and shape of the precursor projections 19 integrally formed along the film or sheet 21 will now be discussed herein. FIG. 3A is an example according to various embodiments illustrating a cross-sectional side view of a precursor projection 19 integrally formed along the film or sheet 21 emerging from the nip 14 in the apparatus 10 of FIG. 1. FIG. 3B is an example according to various embodiments illustrating an end view of the precursor projection 19 formed along the film or sheet 21 emerging from the nip 14 in the apparatus 10 of FIG. 1.

As shown in FIGS. 3A and 3B, the precursor projection 19 has a first portion 39 having a first perimeter 41 (FIG. 3B), a first diameter 42 (FIG. 3A) and a first depth 43 (FIG. 3A) respectively corresponding to the first perimeter 25, the first diameter 26 and the first depth 27 of the first portion 23 of the recess 17. The precursor projection 19 also has a second portion 45 having a second perimeter 47 (FIG. 3B), a second diameter 48 (FIG. 3A) and a second depth 49 (FIG. 3A) respectively corresponding to the second perimeter 31, the second diameter 32 and the second depth 33 of the second portion 29 of the recess 17. As shown in FIG. 3B, the first portion 39 of the precursor projections 19 substantially surrounds the second portion 45 of the precursor projections 19. The first and second portions 39, 45 of the precursor projections 19 are unitary.

FIG. 3C is an example according to various embodiments illustrating a side view of the precursor projection 19 formed along the film or sheet 21 emerging from the nip 14 in the apparatus 10 of FIG. 1. FIG. 3C depicts some example values of various dimensional parameters of the precursor projection 19. These dimensional parameters are not limited to these specific values depicted in FIG. 3C. In some embodiments, the values of the dimensional parameters are the values depicted in FIG. 3C±10%. In other embodiments, the values of the dimensional parameters are the values depicted in FIG. 3C±20%.

As further shown in FIG. 3C, the precursor projection 19 includes a proximal region 51 that is similar to the first portion 39 that is proximate to the plane of the substrate 11. The precursor projection 19 also includes a distal region 53 that is distal from the plane of the substrate 11. As shown in FIG. 3C, in one example embodiment the distal region 53 includes a conical shaped tip.

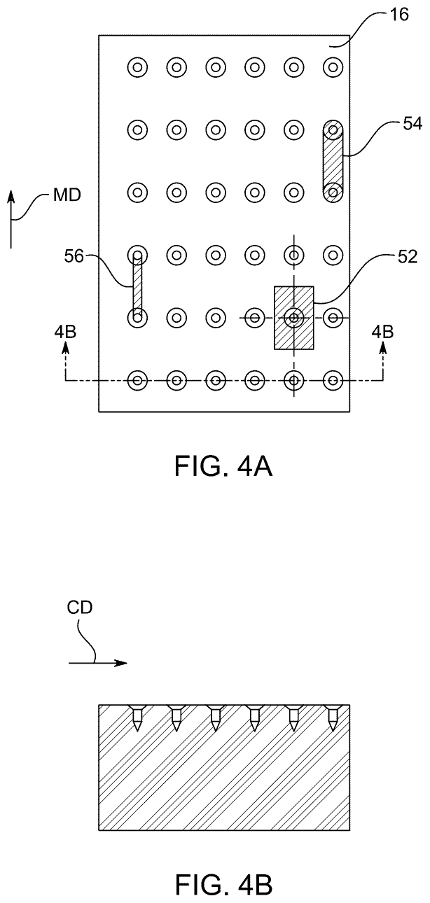

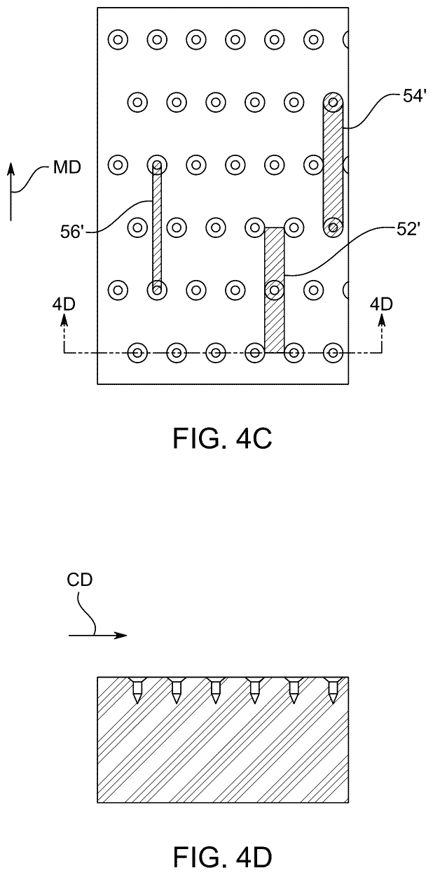

The improvement of the disclosed method that is attributable to the shape and/or dimension of the recesses 17 will now be discussed herein. FIG. 4A is an example according to various embodiments illustrating a top view of the outer surface 16 of the molding roll 15 in the apparatus 10 of FIG. 1. FIG. 4B is an example according to various embodiments illustrating a cross-sectional view of the molding roll 15 in FIG. 4A taken along the line 4B-4B.

As shown in FIG. 4A, an average watershed area 52 is depicted which is based on the total surface area of the outer surface 16 encompassing the recesses 17 divided by the total number of recesses 17. Thus, the average watershed area 52 represents the amount of the total surface area of the outer surface 16 divided equally among the recesses 17. As shown in FIG. 4A, the average watershed area 52 is centered at each recess 17.

As further shown in FIG. 4A, a ported inline watershed area 54 is depicted. This ported inline watershed area 54 is defined by a width and a length, where the width is based on the first diameter 26 of the first portion 23 of the recess 17 in the CD and the length is based on a spacing in the MD between adjacent recesses 17 along the outer surface 16. In other embodiments, where the recesses 17 are non-circular, the width is the CD width of the recess 17.

As further shown in FIG. 4A, a cavity inline watershed area 56 is depicted. This cavity inline watershed area 56 is defined by a width and a length, where the width is based on the second diameter 32 of the second portion 29 of the recess 17 in the CD and the length is based on a spacing in the MD between adjacent recesses 17 along the outer surface 16.

The inventors of the present invention recognized that the ported inline watershed area is indicative of the amount of substrate material that can be passed into the recess 17 during the operation of the apparatus 10. In an example embodiment, the amount of time (e.g. 4-6 milliseconds) at which the substrate material is passed into each recess 17 is quite short and thus the greater the ported inline watershed area, the greater percentage of the average watershed area 52 can be passed into the recesses 17 in this limited time frame. Thus, the inventors of the present invention designed the recess 17 so to increase the ported inline watershed area as compared with conventional recesses. This in turn would improve the method over conventional methods, since the substrate 11 could be moved at a faster speed through the nip 14 and consequently generate more projections over time. Since the first diameter 26 of the first portion 23 of the recess 17 is larger than the second diameter 32 of the second portion 29 of the recess 17, the width of the ported inline watershed area 54 is larger than the width of the cavity inline watershed area 56. Both the ported inline watershed area 54 and the cavity inline watershed area 56 have the same length along the MD. Thus, the ported inline watershed area 54 is larger than the cavity inline watershed area 56, due to the disclosed shape and/or dimensions of the recesses 17. In some embodiments, the ratio of the ported inline watershed area 54 to the average watershed area 52 is indicative of the efficiency at which the substrate 11 material is passed into the recess 17. In an example embodiment, the ported inline watershed area 54 is about 0.6 mm² or in a range from about 0.25 mm² to about 0.9 mm² and the average watershed area 52 is about 0.28 mm² or in a range from about 0.14 mm² to about 0.56 mm². In an example embodiment, the ratio is at least 50% and more specifically at least 70% and still more specifically over 90%. In another example embodiment, the ratio of ported inline watershed area to average watershed area is about 96%, or in a range of about 65% to about 100%.

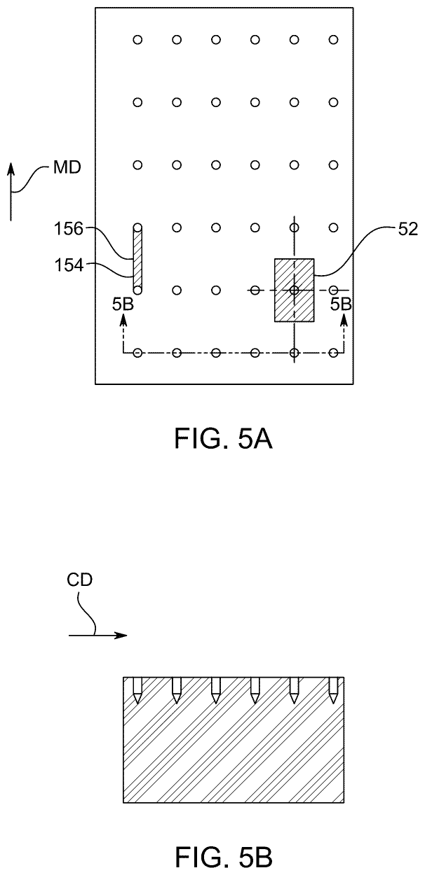

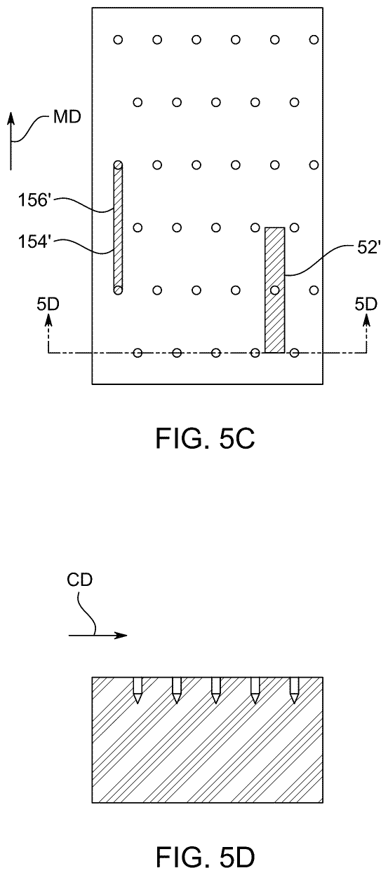

This is in stark contrast with conventional recesses (FIGS. 5A and 5B) that are cylindrical in shape and thus the diameter of the recess is approximately equal along the depth of the recess 17 (e.g. in the first and second portions). Accordingly, as shown in FIG. 5A, the cavity inline watershed area 154 is about the same as the ported inline watershed area 156 for conventional recesses. This is because both the cavity inline watershed area 154 and the ported inline watershed area 156 share approximately the same width (e.g. since the diameter of the cylindrical recess is approximately equal over the depth of the recess) and the same length (e.g. since the MD spacing between adjacent recesses is the same). In contrast with the improved method disclosed herein, the ratio of the ported inline watershed area 154 to the average watershed area 52 is only about 41%.

Although FIGS. 4A and 4B discuss an embodiment where the recesses 17 formed in the outer surface 16 of the molding roll 15 are non-staggered such that consecutive rows of recesses 17 along the MD are aligned in the CD, in other embodiments the recesses 17 have a staggered arrangement, where consecutive rows of recesses 17 along the MD are misaligned in the CD. FIGS. 4C and 4D depict this staggered arrangement of the recesses 17. As with the non-staggered arrangement of recesses 17, the ported inline watershed area 54′ is about equal to the average watershed area 52′. Additionally, the cavity inline watershed area 56′ is much less than the average watershed area 52′. Thus, the staggered arrangement of recesses 17 also discloses a value of the ratio of the ported inline watershed area 54′ to the average watershed area 52′ that is comparable with the above disclosed values with respect to the non-staggered arrangement of recesses 17. Thus, the inventors of the present invention recognized that both the staggered and non-staggered arrangement of recesses 17 advantageously enhances the efficiency of the disclosed method herein relative to conventional methods.

This is in stark contrast with conventional recesses (FIGS. 5C and 5D) for the staggered arrangement of recesses, which are cylindrical in shape and thus the diameter of the recess is approximately equal along the depth of the recess 17 (e.g. in the first and second portions). Accordingly, as shown in FIG. 5C, the cavity inline watershed area 154′ is about the same as the ported inline watershed area 156′ for conventional recesses. This is because both the cavity inline watershed area 154′ and the ported inline watershed area 156′ share approximately the same width (e.g. since the diameter of the cylindrical recess is approximately equal over the depth of the recess) and the same length (e.g. since the MD spacing between adjacent recesses is the same). In contrast with the improved method disclosed herein, the ratio of the ported inline watershed area 154′ to the average watershed area 52 is only about 41%. The inventors of the present invention also recognized other conventional methods (e.g. using a liquid extruder) that are fundamentally different from the method disclosed herein which are used to fill cavities or recesses to generate projections. In such conventional methods, they can increase the amount of material passed into the recesses but does so by increasing the pressure (e.g. from 50 Bar to about 55-70 Bar) on the liquid extruder. The method disclosed herein is fundamentally different from such methods as the method disclosed herein uses material from the substrate 11 itself to fill the recesses 17, rather than filling the recesses with different material separate from the substrate. Thus, a noticeable advantage of the method disclosed herein over these conventional methods which fill the recesses with additional material separate from the substrate is reduced cost, given that the recesses herein are filled with the same existing material from the substrate.

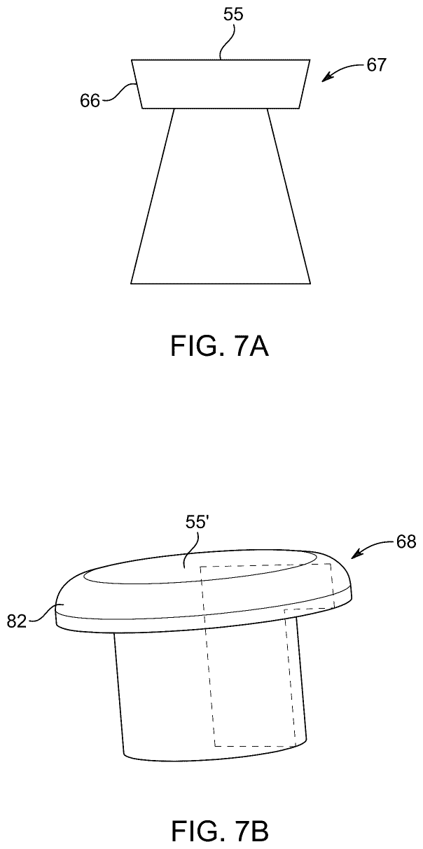

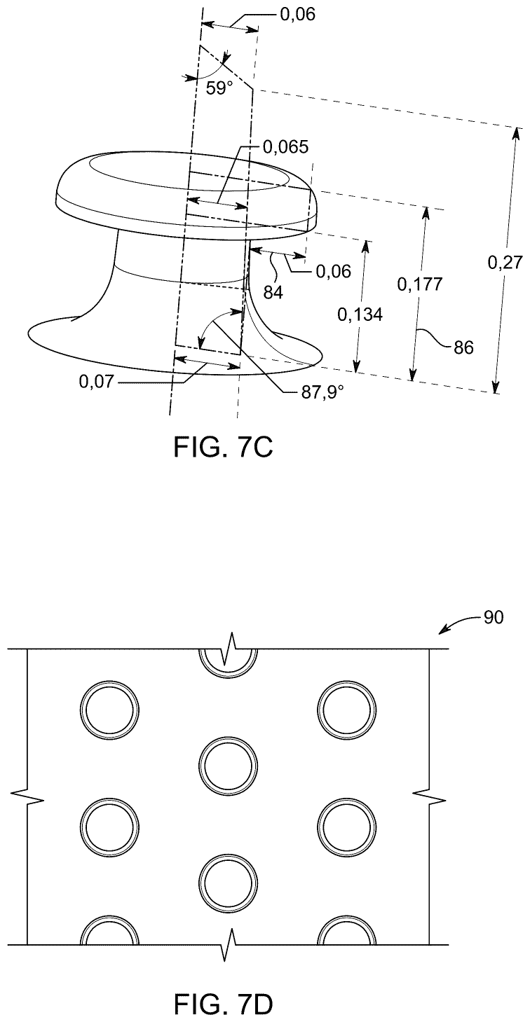

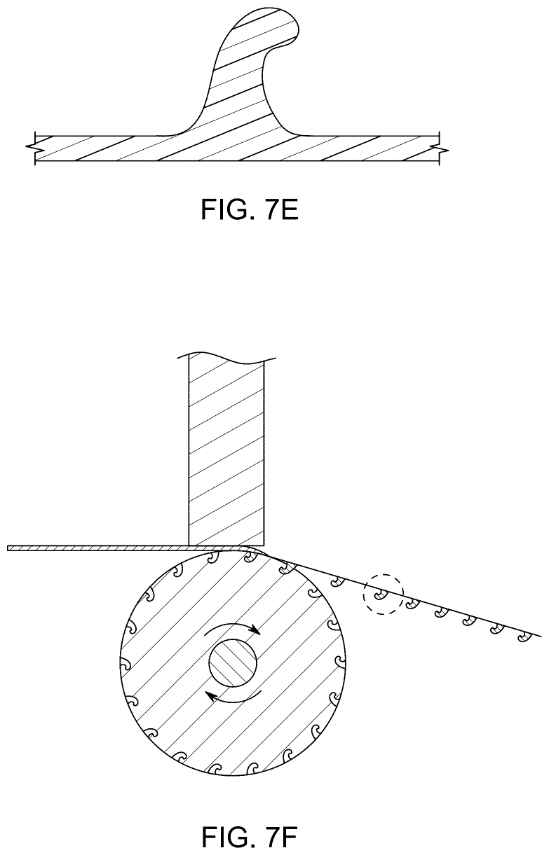

In addition to the ported inline watershed area 52, the inventors of the present invention configured the recesses 17 such that the percentage of the total area of the substrate 11 that is displaced into the recesses 17 by the apparatus 10 is less than a high threshold percentage. In some embodiments, one or more parameter values of the substrate 11 was adjusted, such that this percentage is less than the high threshold percentage. In one embodiment, the substrate 11 thickness after compression was made to be thicker, such that less of the substrate 11 is required to displace into the recesses 17 to fill such recesses 17. In another embodiment, the recesses 11 were made with a smaller diameter and smaller depth, to reduce their volume and consequently reduce the percentage of the total are of the substrate 11 required to fill their volume. In an example embodiment, one or more of the first diameter 26, first depth 27, second diameter 32 and second depth 33 of the recess 17 were adjusted to achieve the desired volume of projection material to form the projection 19 volume having a desired cap and post shape. In this example embodiment, a distal volume of the recess 17 determines cap volume of the projection 19, but increasing the first diameter 26 of the recess 17 consumes more substrate 11 volume without adding to the desired cap volume. In an example embodiment, the adjustment of these dimensional parameters of the recess 17 was performed in order to achieve a shortest, smallest volume projection 19 with enough cap bottom height (first depth 43 of the projection 19) to snag the mating NW and enough cap overhang 84 (FIG. 7C) relative to the post. However, the post diameter (second diameter 48) needs to be thick enough to prevent too much bending and the port diameter (first diameter 26) needs to enable good inflow. In one example embodiment, this high threshold percentage is about 35%. In another example embodiment, this high threshold percentage is about 15%. This enhances the efficiency of the method relative to conventional methods of unitary hook formation (e.g., where the percentage value is about 40%), since this percentage is indicative of a net amount of work that is to be performed in displacing the substrate material into the recesses 17. Hence, the improved method disclosed herein not only maximizes the ratio of the average watershed area 52 that is displaced into the recess 17, which increases the efficiency of the method, it also minimizes the percentage of the total area of the substrate 11 that is to be displaced into the recesses 17, which further enhances the efficiency of the method.

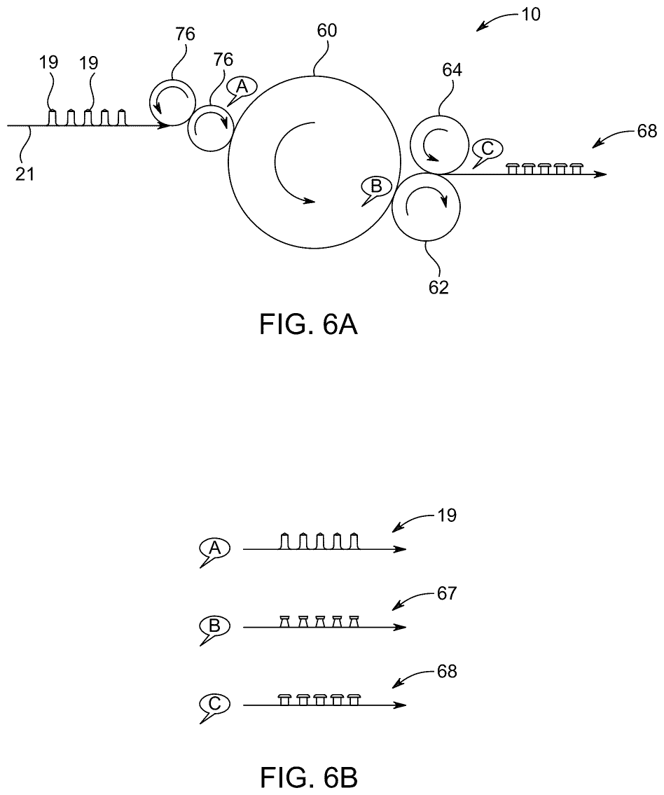

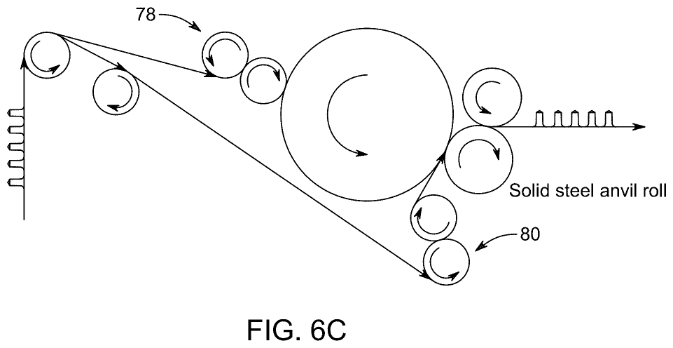

The steps of the method will now be discussed which perform one or more steps on the precursor projections 19 generated by the apparatus 10 of FIG. 1. FIG. 6A is an example according to various embodiments illustrating a schematic view of an apparatus 10 to integrally form projections 68 in the substrate 11 based on an input of the precursor projections 19 formed in FIG. 1. FIG. 6B is an example according to various embodiments illustrating a schematic view of the precursor projections 19, the projections having a flattened distal region 67 and the projections 68 having a mushroom shape at different stages of the apparatus 10 of FIG. 6A.

An initial heating step of the precursor projections will now be discussed. As shown in FIG. 6A, the film or sheet 21 with the precursor projections 19 are incident on one or more idler rollers 76. The film or sheet 21 passes over the idler rollers 76 such that the distal regions 53 of the precursor projections 19 are conveyed over a heated device 60 to heat the distal regions 53. In one embodiment, the heated device 60 heats the distal regions 53 of the precursor projections 19 to a temperature at or above a melting temperature of the substrate 11. In one example embodiment, the heated device 60 is a heated roll (e.g. made from steel or aluminum). In some embodiments, the outer surface of the heated roll is covered with an insulator (e.g. silicon rubber). In other embodiments, the outer surface of the heated roll features intermediate discrete shaped heating sections which are phased to discrete rows of the precursor projections 19 on the film or sheet 21. This advantageously only heats the distal region 53 of the precursor projections 19 and not the substrate or film/sheet 21 between the rows of precursor projections 19. In some embodiments, the heating step is continuous such that the substrate 11 is continuously heated as it moves over the heated device 60. In other embodiments, the heated device 60 is an electric heater with slip rings/inductive coupling or heated fluid is passed through a rotary union or is feedback controlled.

A capping process is now discussed, where the heated distal regions 53 of the precursor projections 19 are flattened. As shown in FIG. 6A, the film or sheet 21 passes over the heated roll so that the distal regions 53 are heated. An anvil roll 62 (e.g. metal anvil roll, such as a solid steel anvil roll) then applies pressure to portions of the distal regions 53 of the precursor projections 19, resulting in a flattened top shaped projections 67 with a flatted distal region 55 (FIG. 7A). Additionally, as shown in FIG. 7A the flattening of the distal region 53 increases the perimeter of flattened distal region 55 relative to the perimeter 47 of the second portion 45 (FIGS. 3A and 3B) of the distal region 53 prior to the flattening step. In an example embodiment, the perimeter of the flattened distal region 55 is greater than the second perimeter 47 of the distal region 53 but is less than the first perimeter 41 of the proximal region 51. The inventors of the present invention recognized that having the perimeter of the flattened distal region 55 greater than the second perimeter 47 of the distal region 53 assists with ensuring a sufficient volume is in the cap so that upon deforming the flattened distal region 55 the resulting projection fulfills certain criteria (e.g. overhang 84 in FIG. 7C). The inventors of the present invention also recognized that having the perimeter of the flattened distal region 55 being less than the first perimeter 41 of the proximal region 51 assists with ensuring not too much volume of substrate 11 fills the recess 17, as this would negatively affect the filling efficiency of the method.

A bumping process is now discussed, where the flattened top shaped projection 67 with the flattened distal region 55 is contacted with a deformable device 64 (e.g. deformable roll). In one example embodiment, the deformable device 64 is a bump roller (e.g. made from silicone rubber). In another example embodiment, the bump roller is loaded against the anvil roll 62 with an adjustable force. As depicted in FIG. 7A, the flattened top shaped projection 67 features an outer portion 66 that is angled upward (away from the substrate 11). The flattened top shaped projection 67 is contacted by the deformable device 64 to deform the outer portions 66 in a direction toward the plane of the substrate 11, resulting in a rounded down outer portion 82 (FIG. 7B). In one embodiment, in the bumping step the deformable device 64 contacts the circumference of the outer portion 66 of the flattened top shaped projection 67. The result of the bumping process is the mushroom shaped projection 68 (FIG. 7B). As shown in FIG. 7B, the flattened distal region 55′ of the mushroom shaped projection 68 is smaller than the flattened distal region 55 of the projection 67 (FIG. 7A), prior to the bumping step.

The apparatus 10 of FIG. 6A can operate in a different mode when the heating step is no longer needed. FIG. 6C is an example according to various embodiments illustrating a schematic view of the apparatus 10 of FIG. 6A where one of the components is moved from a first position to a second position so that the substrate bypasses the heated device 60. As shown in FIG. 6C, one or more of the idler rollers 76 are moved from a first position 78 (FIG. 6A) to a second position 80 such that the film or sheet 21 passing over the idler rollers 76 bypasses the heated device 60. This mode may be selected when the forming of the mushroom shaped projections 68 with the apparatus 10 is complete and thus this mode avoids the substrate 11 coming to a stop on the heated device 60 which may burn through the substrate 11. Thus, in this embodiment the one or more idler rollers 76 are moved to the second position 80 so that the substrate 11 bypasses the heated device 60. In another example embodiment, the one or more idler rollers 76 are moved to the second position 80 for that portion of the substrate 11 between spaced apart patches of the precursor projections 19, since the heating step is not needed for this portion of the substrate 11 between the spaced apart patches. Thus, in these embodiments the heating step is intermittent.

In some embodiments, the capping and bumping process are performed in a similar way as disclosed in U.S. Pat. No. 6,132,660 with the exception of the precursor projections 19 herein which are used as an input to the capping and bumping processes herein as well as intermittent projections (e.g. MD discontinuous and/or CD discontinuous regions spaced apart) or shaped projections. As discussed in more detail below, the values of one or more dimensional parameters of the recesses 17 and generated precursor projections 19 were selected such that when the capping and bumping processes are performed on the precursor projections 19, the mushroom shaped projections 68 are generated with desired values of one or more dimensional parameters. As previously discussed, the method herein is an improvement over conventional methods, as it generates patches of the mushroom shaped projections 68 at a much faster speed than conventional methods and thus enhances the efficiency of the method.

FIG. 7C is an example according to various embodiments illustrating a side perspective view of the projection 68 with a mushroom shape generated by the apparatus of FIG. 6A with one or more dimensional parameters. The dimensional parameters include but are not limited to an overhang 84 of the rounded down outer portion 82 beyond the second diameter 48 (FIG. 3A) and a height 86 of the mushroom shaped projection 68. The values of the dimensional parameters listed in FIG. 7C are merely one example value of these dimensional parameters and the embodiments herein are not limited to these values. In some embodiments, the values of the dimensional parameters are equal to those depicted in FIG. 7C ±10% and/or ±30-40%. In other embodiments, the dimensional parameters (e.g. height of the projection) are equal to those depicted in FIG. 7C ±60%.

In some embodiments, the mushroom shaped projections 68 generated by the method disclosed herein are suitable for use as a portion of a touch fastener. In an example embodiment, this touch fastener is used for a primary fastening tab of a taped diaper, a secondary fastening region of a taped diaper, a disposal tape of a taped or pant diaper, a primary fastening patch of a pant diaper, a pre-closed and/or refastenable primary fastening patch of a pant diaper, a disposal tape of a pant diaper, an attachment patch for an absorbent core in a diaper, feminine hygiene article, adult incontinence article, or similar uses.

FIG. 7D is an example according to various embodiments illustrating a top view of a shaped patch 90 of formed projections 68 in the substrate using the apparatus 10 depicted in FIGS. 1 and 6A. In some embodiments, the method disclosed herein is used to form a shaped patch 90 of the fully formed projections 68 in the substrate 11.

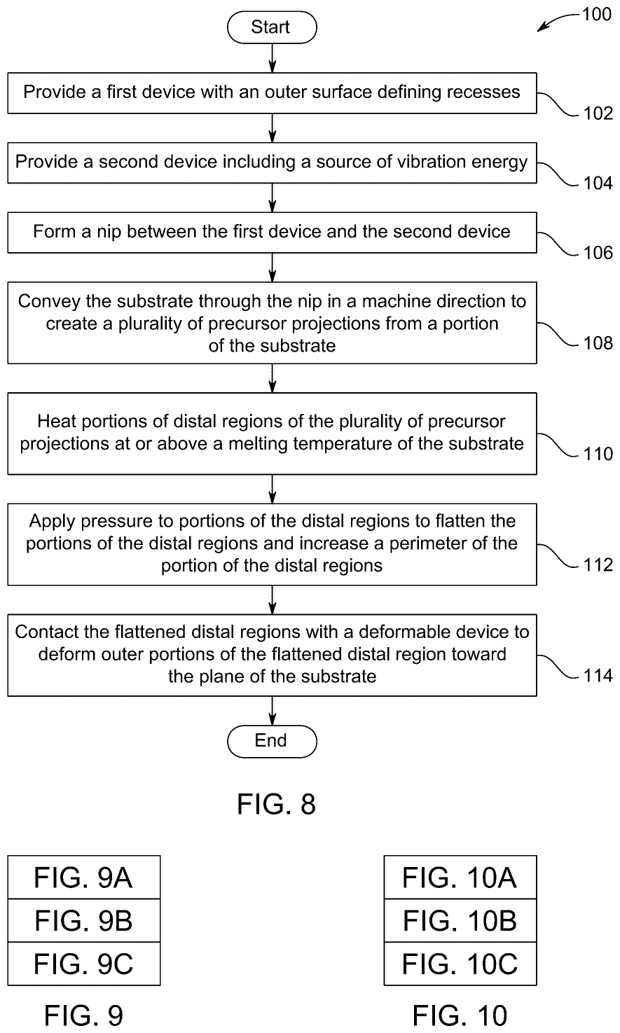

A flowchart of the previously disclosed method will now be discussed. FIG. 8 is an example according to various embodiments illustrating a flowchart depicting a method for integrally forming projections in a substrate. Although steps are depicted in FIG. 8 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 102, the first device (e.g. molding roll 15) is provided with the outer surface 16 that defines the plurality of recesses 17. The example embodiment section below discusses various techniques that can be employed to form and shape the recesses 17 in the outer surface 16.

In step 104, the second device 13 (e.g. source of vibration energy, such as a blade sonotrode or rotary sonotrode; a source of heat or pressure, etc.) is provided.

In step 106, the nip 14 is formed between the molding roll 15 and the second device 13.

In step 108, the substrate 11 is conveyed through the nip 14 in the MD. During this step, the second device 13 is activated which generates vibration energy that causes material from the substrate 11 to pass into the plurality of recesses 17 along the outer surface 16 of the molding roll 15. This causes the plurality of precursor projections 19 to be integrally formed on the sheet or film 21 emerging from the nip 14. In this embodiment, step 108 results in the sheet or film 21 with the plurality of precursor projections 19.

In step 110, a portion of the precursor projections 19 are heated. In one embodiment, in step 110, the distal regions 53 of the precursor projections 19 are heated at or above a melting temperature of the substrate 11 material. In one embodiment, the heating in step 110 is performed by the heated device 60 (e.g. heated roll) over which the distal regions 53 of the precursor projections 19 are conveyed using the idler rollers 76.

In step 112, pressure is applied to the heated portions of the precursor projections 19 from step 110. In one embodiment, in step 112, pressure is applied to the heated distal regions 53 from step 110. In one example embodiment, in step 112, pressure is applied by the anvil roll 62 resulting in the flattened top shape projection 67 having the flattened distal region 55 (FIGS. 6B, 7A). Additionally, in one embodiment, in step 112 the applied pressure to the heated distal regions 53 of the precursor projections 19 by the anvil roll 62 cause a widening of a perimeter of the flattened distal region 55 as compared to the perimeter of the previously unflattened distal region 53.

In step 114, the flattened distal region 55 of the flattened top shape projection 67 is contacted with the deformable device 64. In one embodiment, in step 114 the contact of the flattened distal region 55 with the deformable device 64 deforms outer portions 66 of the flattened top shape projection 67 towards the substrate 11, resulting in a rounded down outer surface 82 (FIG. 7B). In an example embodiment, the contacting in step 114 is performed over the circumference of the flattened distal region 55 such that the rounded down outer surface 82 is provided over the circumference of the mushroom shaped projection 68.

### Method Example Embodiments

Some example embodiments of the previously disclosed method will now be discussed herein.

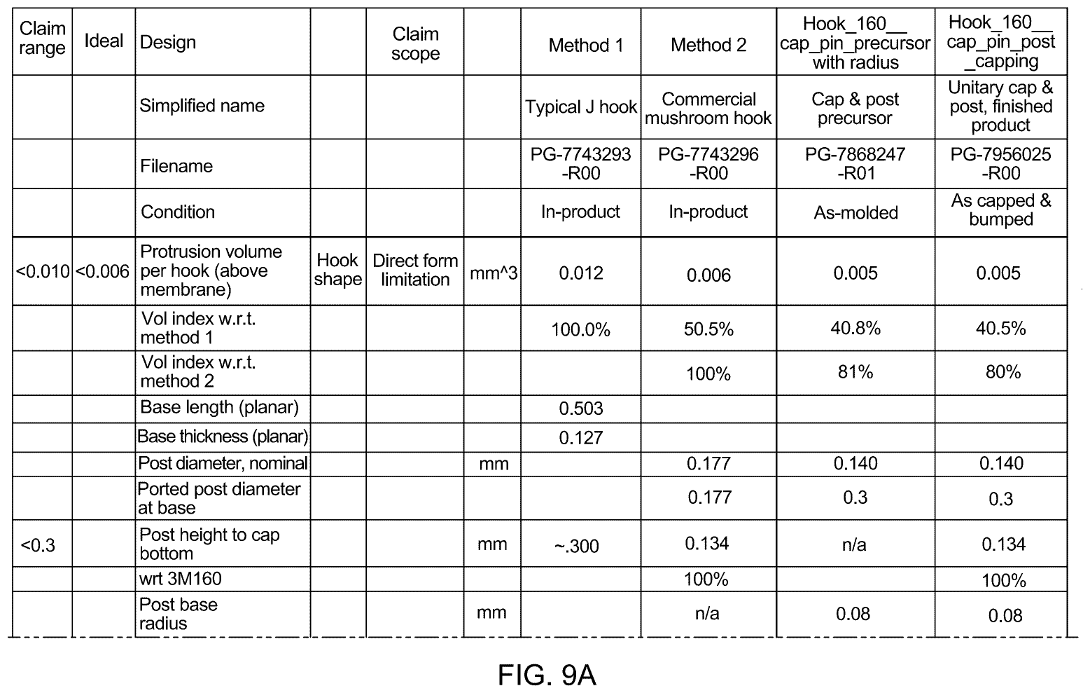

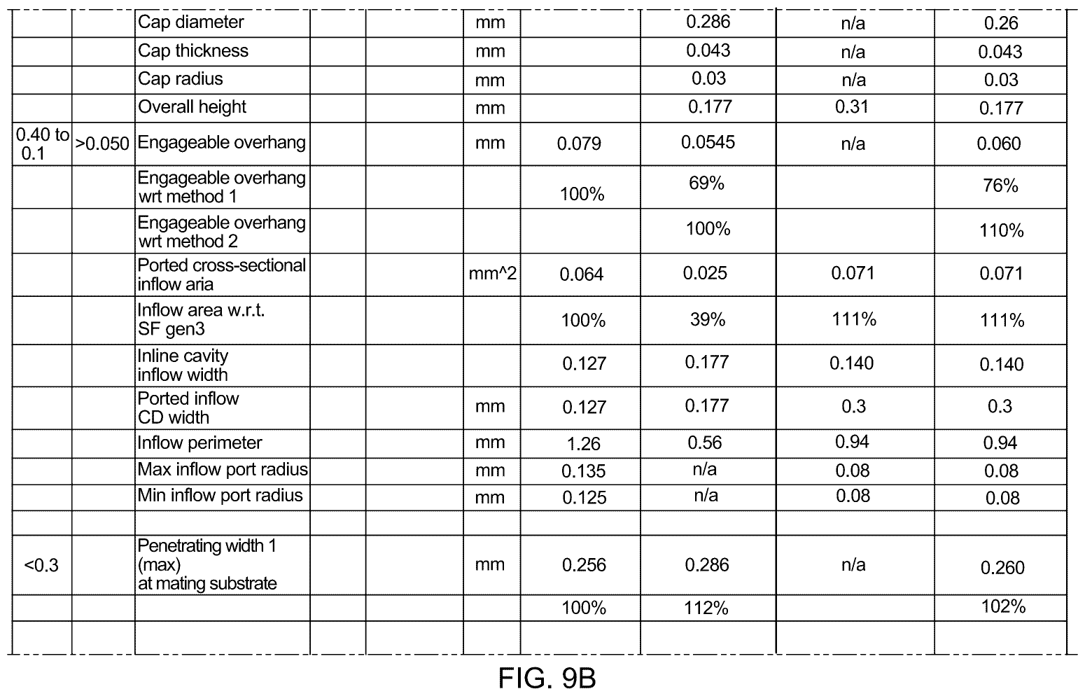

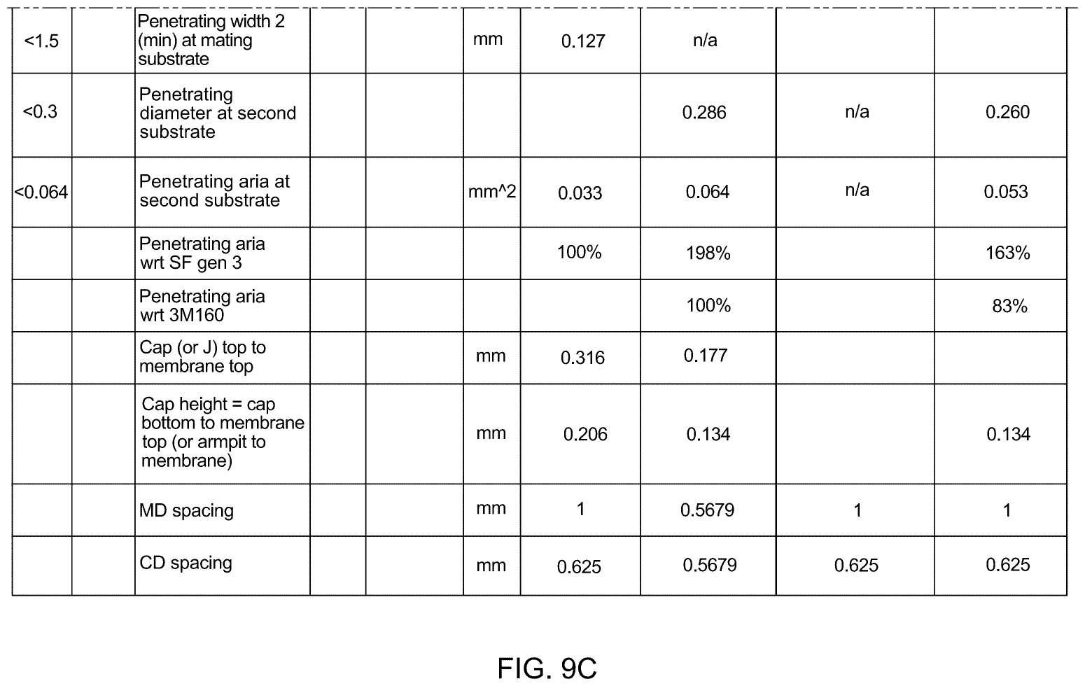

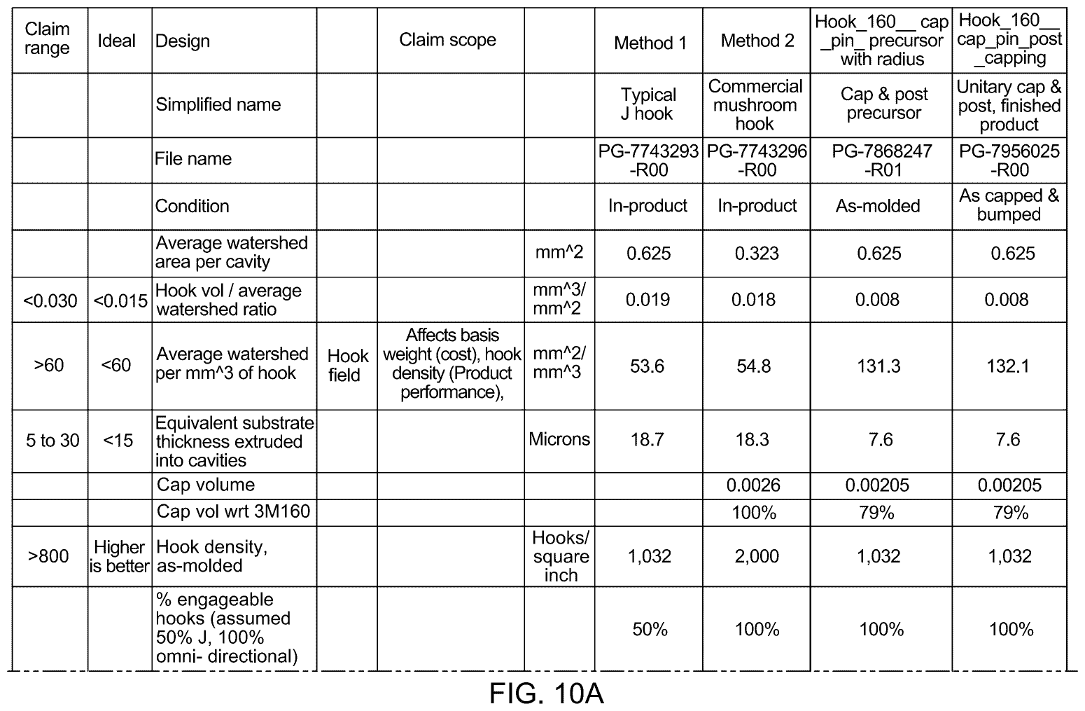

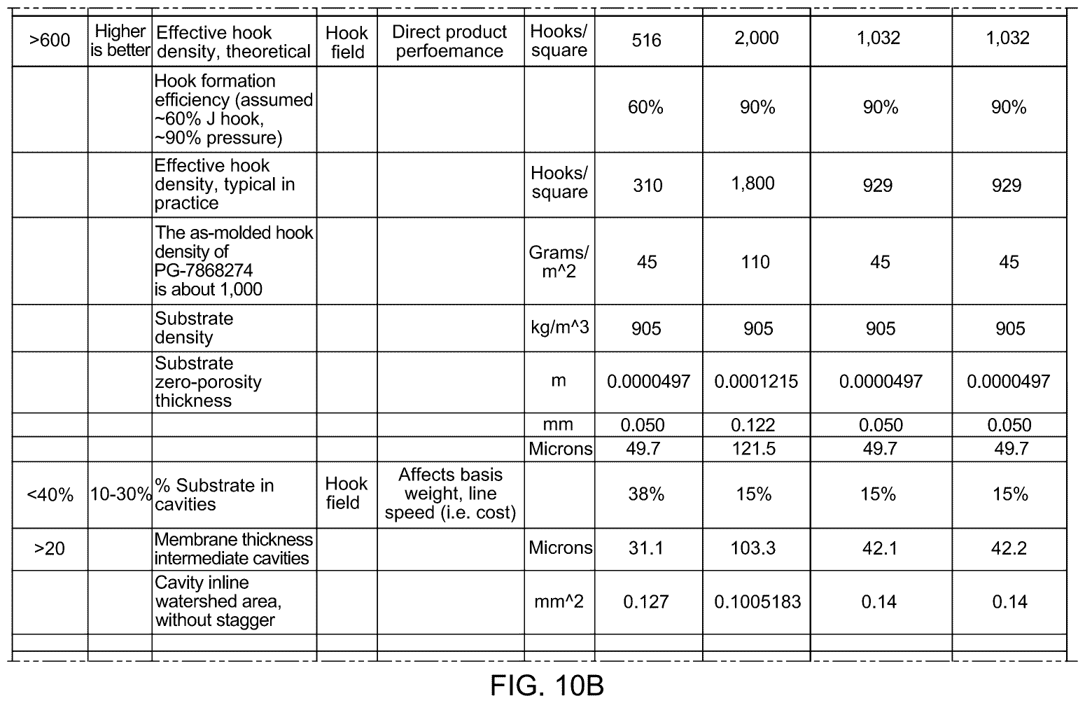

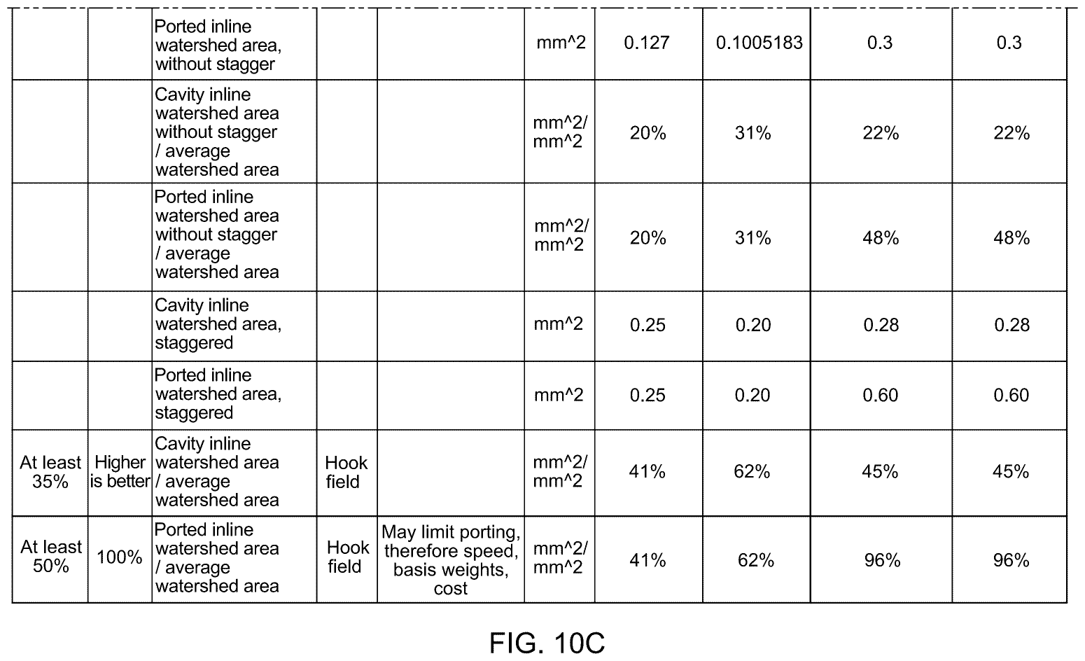

FIGS. 9A-9C and 10A-10C list some example values for some parameters for projections generated by two conventional methods (“Method 1”, “Method 2”) as well as parameters for the precursor projections 19 (“Hook_160_Cap_Pin_PRECURSOR”) and parameters for the mushroom shaped projections 68 (“Hook_160_Cap_Pin_Post_Cappling”). The values listed in FIGS. 9A-9C and 10A-10C for these dimensional parameters are merely values for one example embodiment of the method herein and thus these dimensional parameters are not limited to the specific values listed in FIGS. 9A-9C and 10A-10C. In some embodiments, the values of the dimensional parameters are based on the values listed in FIGS. 9A-9C and 10A-10C ±10% and/or the values listed in FIGS. 9A-9C and 10A-10C ±20%.

Some values of the parameters listed in FIGS. 9A-9C and 10A-10C will now be discussed. As shown in FIGS. 9A-9C, the value of the protrusion volume per hook is only 0.005 mm³ for the precursor projections 19 and mushroom shaped projections 68 whereas the value of this parameter for one of the conventional methods is 0.012 mm³. Smaller projection volumes make the recesses easier to fill. A small recess requires less substrate to be displaced in CD and/or MD during the near instantaneous nip process. Another parameter value of interest is the average watershed area per mm³ of hook. As shown in FIGS. 9A-9C, the value of this parameter is about 55 mm²/mm³ in the conventional method whereas the value of this parameter for the projection protrusions 19 and mushroom shaped projections 68 is about 131 mm²/mm³. Said differently, a larger watershed area feeding into each recess means the recess is easier to fill. The ratio of watershed area to volume of the recess enables a figure of merit for comparison across different cavity volumes. In energy terms, a larger ratio of watershed area to recess volume enables a higher energy density from the sonotrode or other vibrational source.

The inventors of the improved method disclosed herein recognized that the recesses 17 and thus the precursor projections 19 should have the proper shape and/or dimensional parameter values in order to achieve certain desired outcomes with the method. For example, the inventors recognized that if the base port hole (e.g. diameter 26 in FIG. 2A) is too wide, this will necessarily increase the volume of the precursor projections 19 in the first portion 39 and thus potentially decrease the volume of the precursor projections 19 in the second portion 45 (FIG. 3A) below a threshold value that is needed to achieve the desired mushroom shape projection 68 after the capping and bumping steps 112, 114 are performed. Thus, the inventors designed the recesses 17 with the proper geometry (e.g. dimensional parameter values disclosed herein) such that the right amount of substrate forms each of the first and second portions 39, 45 of the precursor projection 19 so that when the capping and bumping steps 112, 114 are performed, the result is the mushroom shape projections 68 with the desired parameter values disclosed herein.

In addition to the drawbacks of the conventional methods previously discussed herein, other conventional methods generate planar hooks which have several shortcomings. Such planar hooks, as shown in FIG. 7E, may have low hook count density (e.g. few hooks per square inch). Such hooks may have low peel or shear bond strengths in finished product. As further shown in FIG. 7F, fully formed planar hooks may not extract easily from mold rolls, which may lead to process limitations such as low production rate and/or hook shapes which are non-desirable from product point of view. Planar hooks made by unitary formation may have the disadvantage of a small and/or narrow cavity opening adjacent a membrane region. It may be difficult to achieve sufficient substrate material flow into the cavity during the few milliseconds that each portion of substrate passes through nip point. The narrow width of each of the individual disk in a stack may aggravate this issue. Rolls comprising a plurality of disk elements may be susceptible to dimensional instability, such as excessive runout, relative motion of disks, or permanent deformation of portions of the disks and/or roll after use.

A solution to the disadvantages of planar hooks for unitarily formed fasteners is to use a cap and post style hook (e.g. mushroom shaped projection 68). An advantage of the cap and post style hook may be that the cavities (e.g. plurality of recesses 17) in the molding roll 15 need not directly form the hook element. Instead, a plurality of smooth protrusions (e.g. precursor projections 19) such as cylinders or pins are formed on the substrate 11 via said mold. Substrate flow into the plurality of cavities may be improved by the simple cavity geometry and porting or rounding at the entrance to the cavities. As-molded protrusions may extract more easily from a plurality of mold cavities than the finished fastener shape after capping and/or bumping. Such simplifications may increase throughput, eliminate complex machining, enable a higher density of hooks, and/or enable lower basis weight materials. This as-molded post (e.g. precursor projections 19) serves as the base for the hook formation. A hook, in this context, refers to any structure with a surface that is displaced from a planar substrate and serves to provide a constraining and/or locating function for a fiber of a mating second substrate.

After forming multiple precursor protrusions such as cylinders or pins, the substrate may be extracted from the mold roll using a stripper roll. The tips of the pins may be deformed to create a cap, such as mushroom shaped projection 68. This cap may be circular, rounded, or consist of a plurality of petals or smaller hooks. An example cap formation process is disclosed in prior art, such as U.S. Pat. No. 6,132,660. Capping may utilize a heated roller to preferentially heat the distal tips of the hooks. Under pressure from a steel-to-steel roll or another hard surface, these heated tips may be deformed into a cap shape. Additionally, as disclosed in prior art (U.S. Pat. Nos. 6,132,660, 5,679,302, 5,505,747) the hooks may also be rounded over utilizing a second bumping process, such as a silicone roll pressed against the distal end of the capped hooks. This bumping process may offer aesthetic benefits, such as a softer feel, and may enhance performance by allowing the hook shape to better penetrate and engage with materials like woven fabric, forming a hook and loop fastener.

In summary, the cap and post style hooks present a novel solution to overcome the limitations of planar hooks for unitarily formed fasteners. Through an ultrasonic formation process and the utilization of a mold roll, a cylindrical post (e.g. precursor projections 19) is created, which serves as the foundation for the hook formation. The tips of the hooks (e.g. distal regions 53) may be deformed into a cap shape, and subsequent processes, such as heating and rounding, may be applied to refine the hook's characteristics. This innovative approach offers both aesthetic and performance advantages, making it suitable for applications where a secure and reliable hook and loop fastening system is required.

There may be several constraints in the throughput rates of traditional stacked disk or planar style hooks. Cap and post hooks may withdraw more easily from a mold roll than alternate hook designs. For example, some planar, J-shape, or T-shape hooks may have a hook geometry which tends to kinematically trap the hook distal end in the mold roll. Said hook geometries may require substantial deflection and strain in the as-molded shape for the hook to pull out of the cavity during extraction. Such high deformation, or mechanical strain, may cause high in-process mechanical stresses and may lead to fracture of hooks during extraction. Said hooks may not pull out mold roll cleanly and may clog mold rolls. This fracture may occur at the base due to weak bonds between the hook and the planar substrate region. Such fracture may be caused by insufficient basis weight of material. Such fracture may be caused by insufficient fiber to fiber bonding, such as in the case of a hook made from a nonwoven substrate where all the individual fibers must be bonded together into continuum solid hook. Such fracture may be caused by poor mold roll filling, such as when a nonwoven at high speed is displaced into a hook cavity and a portion of the displaced NW does not fill the cavity completely. Such fracture may be caused by poor mold roll filling due to the lack of a draft angle in the hook cavity shape.

Cap and post hooks (e.g. precursor projections 19) may be more easily withdrawn from a mold roll than prior art planar hooks. Planar hooks may commonly have a J shape or T shape in prior art. Such J or T shaped hooks must bend, often to very high localized strains, to extract from the mold cavity. Due to the high strain, such hooks may often fracture during extraction. This may lead to damage during fabrication as the hooks do not cleanly detach from the mold roll and may fracture instead. Fractures can occur at the base of the hook due to poor bonding between the hook and the substrate. This can be caused by insufficient basis weight material or inadequate fiber-to-fiber bonding in the case of non-woven hooks where individual fibers need to be bonded together. Poor mold roll filling, such as incomplete cavity filling when a non-woven material is displaced into a hook cavity at high speed, or the absence of a draft angle in the hook shape itself, can also contribute to these issues. These factors may result in plugged mold rolls, lower hook density in the finished product, and rough or poorly formed hooks, which not only have aesthetic implications but may also reduce the product's performance in terms of plate shear or peel forces.

Prior art hooks which do not extract cleanly from a mold roll may lead to plugged hook cavities, leading to a reduced percentage of hooks which successfully form versus the actual quantity of hook cavities in a mold roll. For example, in hook designs from conventional methods, as few as 40%, 50%, 60%, or 70% or 90% of the hook cavities may successfully form hooks. The percentage of hooks which form relative to the number of mold roll cavities may be defined as the “hook formation efficiency”. Said plugged cavity failure mode of conventional mold rolls, poor mold cavity filling, said fracture of hooks and/or contamination of the mold roll with foreign substances may result quality defects. Examples of quality defects are fewer hooks per square inch in the finished product, poorly or incompletely formed hooks in the in the finished product. Such defects may be aesthetically unpleasing and/or may lower the technical performance of a hook region. Technical performance metrics may comprise peel forces, shear forces, plate shear forces, and/or dynamic shear forces. Such technical performance metrics may apply in a first direction and/or a second direction. A esthetically unpleasing factors may comprise a visual appearance, non-uniformity of a hook field of a plurality of hooks (e.g. splotchy), sharp tactile distal ends from partially formed hooks, tactile rough edges from hook sites where a hook has fractured from a planar substrate, and/or holes in a substrate containing a hook field. Such examples of technical and aesthetic parameters are non-limiting. In contrast, cylindrical pin hooks (e.g. precursor projections 19 disclosed herein) may extract from a mold with almost no forces. Cylindrical as-molded protrusions may have a slight draft angle, about 0.5° to about 10°. Cylindrical as-molded protrusions may have lower strain during extraction.

In an example embodiment, some suitable machining methods used herein to form the recesses include but are not limited to: laser ablation, wire EDM (electro-discharge machining), plunge EDM, acid etching, photo-etching, corrosive machining, micro milling, and/or electron beam drilling.

Regardless of machining method, a plurality of cavities may be created in regions or the entirety of a circumferential surface of a cylinder. Hooks fields may be grouped in patches, MD region, CD regions, complex shapes of hook fields, or hook fields shaped as icons.

It may be quite beneficial to have a rounded entry at the circumferential edge of any of the hook cavities (e.g. recesses 17). For example, such edge shape may be rounded and/or chamfered into the cavity itself. Such porting may aid fluidic material flows into the hook cavity. The substrate material may be a liquid phase during this flow. The substrate may be a solid phase during this flow. The substrate material may be a solid phase under post yield deformation or plastic deformation during this flow.

The wetted area of the cavity opening intersecting or adjacent to the membrane surface (e.g. area encompassed by the perimeter 25 in FIG. 2B) may be larger in the cap and post hook versus planar hooks, providing the advantage of the wider opening and thus more easier material flow. For example, a 0.160 mm diameter hook base (e.g. diameter 42 in FIG. 3A) of a cylindrical pin may intersect 0.160 mm of CD width of substrate 11 at the in-running nip 14 region between a sonotrode 13 (rotary or blade) and a mold roll 15. Prior art planar hooks may be only 0.127 mm wide in CD, presenting an increase of over 25% in area. Where the cavity is radiused by 40 microns at the circumferential intersection of exemplar cylindrical cavity and circumferential surface, the effective cross direction width may be 0.260 mm (160+40+40), representing a significant >200% increase in cross-section width relative to a planar J-hook of the same cavity volume.

For the same example, a 0.160 mm diameter cap and post cylindrical pin may have a cross sectional flow area of 0.020 mm² at the base. A similar planar J-style hook of 0.127 mm CD by 0.300 mm may have a cross sectional area of 0.381 mm². The planar hook may initially have a greater flow area. By radiusing the cylindrical pin at the base, the cross sectional area funneling fluidic substrate into the cavity may be increased to 0.260 mm diameter, yielding a flow area of 0.530 mm², which is substantially larger than a planar hook. Planar hook cavities may be difficult or costly to radius along the CD edges of the spacer disks.

A hook volume to port area figure of merit may be defined as a performance metric. In the prior art example above, the hook volume was about x₁ mm³ per y₁ mm² of port area, or a ratio of x₁/y₁. In the disclosed method herein, the hook volume is x₂ mm³ per y₂ mm² of port volume, or a ratio of x₂/y₂. A lower ratio of hook volume to port area may indicate improved flow into the mold cavity and/or improved extraction from the mold cavity.

A port area to watershed area ratio may be established as a figure of merit. In the prior art example above, the port area x₁ mm² per z₁ m² watershed area, or a ratio of x₁/z₁. In the novel example above, the port area x₂ mm² per z₂ m² watershed area, or a ratio of x₂/z₂. A higher ratio of port area to watershed area may improve flow into the mold cavity and/or improve extraction from the mold cavity.

Such porting may improve extraction of hooks. For example, having a cylindrical post with a sharp 90° corner at the intersection with a planar membrane, there may be very severe stress concentrations proximate the intersection of hook post and planar membrane base. A hook may be likely to break at these stress concentrations during very fast extraction from the mold roll. By providing a rounded mold shape, the stress concentrations may be reduced proximate the post-membrane interface. The mechanical stresses during extraction may be highest in this region.

In some embodiments, a first rounded shape may be utilized on a leading edge of a hook cavity and a second rounded shape may be used on a trailing edge. Such shapes may be the same radius or different radii. Such shapes may be machined by a ground tool, which may have a complex curved shape. Such porting may be created via a first and second tool, or via rotating a first tool around a major axis of said mold roll.

The trailing edge of the cavity may have a discontinuous cutout or channel. Such cutout may provide a space for outflow of material along a trailing edge. Such outflow may ensue good mixing of polymers on the trailing edge of the hook cavity. In prior art without such cavities may lead to flow stagnation and inadequate mixing of polymers in this region, which may result in hook fracture at extraction from the mold roll.

Any of these fabrication techniques may be implemented as a plurality of tooling inserts, where said tooling inserts may be mounted on a drum, roll, or shaft. Any of these fabrication techniques may be implemented as cavities in one or more rings, where said rings may be mounted on cylindrical roll. Any of these fabrication techniques may be implemented as cavities on a wider cylinder, drum, shaft, endless belt, or roller.

The cap and post method disclosed herein may provide the process benefit of enabling higher heat. It has been found by the inventors that higher substrate temperatures may provide a substantial benefit to hook and/or protrusion formation in unitary formed fasteners. For example, prior art models may be in the range of 38° C. to 50° C. before thermal stability problems manifest. Failure modes may comprise shifting of disks or warping of disks as previously discussed.

Cap and post mold rolls also offer initial benefits from a heat perspective. Preheating the substrate has been observed to be highly effective in achieving higher throughput and better quality hooks. Preheating enhances bonding between fibers and the unitary form fastener, resulting in stronger hook attachment forces to mating nonwoven materials. However, current preheating embodiments are limited by the mold roll's tolerance for high temperatures. In prior art, mold rolls are typically limited to preheat temperatures below 50 degrees Celsius. However, by utilizing a heat-resistant mold roll, hot air can be directed at the substrate, such as an adhesive film or laminate, and the mold roll itself. This maintains the material at its peak temperature adjacent to the nip point. Substrate temperatures can be significantly higher, such as in the range of 160 to 250 degrees Celsius for preheating a polypropylene nonwoven. By preventing the substrate from cooling between the preheating step and the nip point, maximum throughput and quality can be achieved. For example, if a substrate heated to 140 degrees Celsius cools off by 20 degrees Celsius before reaching the nip point, it will only provide the benefits of a material softened to 120 degrees Celsius. However, if the preheat can be maintained right up to the nip point, the apparent viscosity and/or elastic modulus of the substrate at the formation point can be substantially lowered. This improves filling and bonding of fibers, resulting in significant economic benefits for the fabrication process. In prior art, such high preheat temperatures may damage mold rolls and may not be feasible with cylindrical drum rings or monolithic inserts. However, such concerns can be eliminated with cap and post hook style mold rolls. Not only are these mold rolls dimensionally stable under high force, but they also exhibit dimensional stability under thermal excitation.

A cap and post hook may utilize a mold roll with a solid, monolithic surface in the working tool area. A solid surface may enable heating to a more advantageous temperature of within 60 degrees Celsius to 40 degrees Celsius or to within 10 degrees Celsius or to within 5 degrees Celsius or to within 1 degree Celsius of the melting temperature of the substrate. For example, for a polypropylene which melts at 162 degrees Celsius said polymer may exhibit softening in the range of 120 to 160 degrees Celsius. Without being limited by theory, softening may be due to the melting of a portion semi-crystalline domains within a substrate polymer into amorphous domains. At the final melting temperature, 162° C. in our example, all semi crystalline domains may convert into amorphous material. By reducing the semi-crystalline domains, the Young's modulus and the apparent viscosity of the material may be substantially reduced. For example, the Youngs modulus may be reduced by a factor of 3×, 4× or 5×. For example, the apparent viscosity may decrease by up to an order of magnitude or more when the material is heated. High shear stress and or elevated temperature may enable better filling of the mold roll cavity. It may be surprisingly found that a softened thermoplastic more easily fills a mold cavity more easily than a hard and rigid thermoplastic. As a process benefit, these methods may enable substantially increased line speeds. Increase line rate and/or lower cost mold roll fabrication may provide a more capital efficient production system. Further, mold rolls with a plurality of cavities maybe substantially lower capital cost than prior art such as the Velcro stacked disk rolls.

When considering the product volume rounded corners at the base of the protrusion may be preferred from both a hook filling and a stress concentration in finished product. From a hook filling perspective this rounded shape need not be symmetrical at leading and trailing edges of the hook. A leading edge radius may be chosen for improved polymer flow into a mold. A trailing edge radius may be chosen for good mold extraction and/or to minimize the volume of polymer consumed. For example, in prior art planar style hooks poor mixing of material resulting in fractures intermediate fibers has been observed by the inventors. Such fractures may limit product forces and mold speeds. In some embodiments the corner element adjacent the membrane surface may be asymmetrical. For example, the intersectional region of a protrusion and a membrane may be a rounded conical shape or parabolic or a rolled parabola shape. In other embodiments an additional cut out at the trailing edge may be used to ensure substrate flow, which may be turbulent, past the trailing edge of the intersection between post and membrane. In prior art, there may be stagnation point in the polymer flow at the trailing edge of the protrusion.

Cap and post mold rolls may enable more efficacious heating of tooling and/or substrate. Substrate Preheat has been observed to be effective in enabling higher throughput and higher quality hooks. Substrate preheat has been observed to provide higher hook attachment forces to a mating nonwoven. Without being limited to theory, substrate preheat may improve bonding between precursor fibers within the unitary formed fastener, enabling a stronger hook. Current preheating embodiments maybe limited by the tolerance of the mold roll for high temperatures. For example, mold rolls in prior art may be preferentially less than 50 degrees Celsius to prevent equipment damage. Preheat temperatures for air preheating of a nonwoven of a polypropylene nonwoven may be on the order of 160 degrees Celsius to 250 degrees Celsius. By enabling a heat resistant mold roll, thermal energy may be directed proximate the nip point between mold roll, substrate, and/or sonotrode such that the material is maintained at its peak temperature proximate the nip point. The temperature of the mold roll may be above 50° C., above 90° C., above a Vicat softening temperature of a substrate, above 120° C., above 140° C. and/or below a melting temperature of a substrate. Prior art mold rolls may exhibit a circular indicated runout of 8 microns, 15 microns or even 20 microns after heating above 35° C. A target circular indicated runout for an ultrasonic formation process may be about 5 microns (0.005 mm). The method disclosed herein provides this capability despite heating, which may be via eliminating regions of differential thermal expansion in the mold roll and/or eliminating relative motion between disks in a mold roll.

High substrate temperatures may be limited by the substrate properties. For example, a polypropylene material which melts at 162 degrees Celsius may be limited to 140 degrees or 150 degrees Celsius. Such limitation may be due to negative process transformations, which may comprise formation of holes and wrinkles inside material, shrinkage of the substrate in one or more direction, and/or loss of substrate softness. Preventing the substrate from cooling between the preheating step and the nip point may be important for maximizing throughput and product quality. For example, a substrate heated to 140 degrees Celsius which cools off by 20 degrees Celsius before between the Preheat point and the net point would only provide the throughput benefits of a material softened to 120 degrees Celsius. Given the low basis weight of typical disposable absorbent article substrates, said substrate may cool as much as 15-40° C. in only 10-40 mm at substrate velocities of about 30-70 m/min. If instead the substrate can be maintained at 140° C. up to the nip point, the apparent viscosity and/or elastic modulus of the substrate at formation point may be substantially lowered. Filling and bonding of fiber to fiber may be improved. This improved pre-heat may provide great economic benefits to the fabrication process. In prior art such preheat may incidentally heat and damage mold rolls and thus may not be feasible. With the cylindrical drum ring or monolithic insert disclosed herein, such concerns may be obviated. Not only are such cap and post hook style mold rolls dimensionally stable under high force they may also be dimensionally stable at higher temperatures. Prior art mold rolls may become damaged at low forces of about 2-3 kN per 20 mm of CD pattern width. Monolithic mold rolls may enable compression forces of 3, 4, 5 kN, or more per 20 mm of product with. In an example embodiment, the actual product of the disclosed method herein is a shaped discrete patch of about 20-45 mm CD width.

When considering the product's geometry, the presence of rounded corners at the base of the hook protrusion offers advantages in terms of both hook filling and stress concentration in the finished product. These rounded corners need not be symmetrical and can take various shapes. For instance, in prior art planar style hooks, poor mixing of material has been observed, resulting in fractures between intermediate fibers. Such fractures can limit product forces and mold speeds. In certain embodiments, the corner element adjacent to the membrane surface can be asymmetrical, resembling a rounded conical, parabolic, or rolled parabola shape with an additional cutout at the trailing edge. This cutout can take the form of a small channel at the downstream edge, allowing flow to pass beyond the region where the hook is adjacent to the membrane. This ensures that any flow stagnation point with reduced mixing occurs outside the critical bonding area. This technique is observable in finished product.

The capping operation will now be discussed. In prior art U.S. Pat. No. 6,132,660, a micro replication process is disclosed. Said micro replication process comprises forming a plurality of posts on a substrate utilizing a thermal mold, without the use of ultrasonics, and then transporting said substrate with a plurality of protrusions through a first a steel-steel nip point called the capping operation. Said capping operation transforms the plurality of cylindrical protrusions into a plurality of flat topped cylindrical protrusions with a larger cross-sectional region proximate the distal ends of the protrusions. These may commonly appear as T shape in cross section, or as a mushroom shape. Other prior art discloses transporting said T shaped cross section, or mushroom, hooks through a set of rubber-to-steel or rubber-to-rubber rollers. The deformable rubber element may round over the top of the flat top hooks so that they have more of a traditional mushroom shape. This bumping step may provide benefits such as a soft hand feel for the wearer. Said product with a bumped cap may provide performance benefits, such as better penetration of the mating non-woven and therefore higher engagement forces. The conical shape may push NW fibers aside as the hook penetrates the network of NW fibers, whereas flat topped hooks may be less likely to penetrate a NW.

In prior art the heat energy for such capping and bumping operations as they are known may be from the initial molding process. For example, a thermal mold roll at elevated temperature is used for example in the prior art process. The molding operation may be at or near a melting temperature for a substrate, such as near 160 degrees Celsius for a polypropylene. Due to short transport times the material may still be at all elevated temperature, above its softening temperature, when it transits the nip points of the caping roll and/or the bump rolls. Said capping and bumping rolls may be heated.

In the improved method here, where the cap and post unitary form fasteners, this thermal energy may not be present or may not be present to the same degree. For example, it has been observed that polypropylene substrate molded via the ultrasonic formation process, results in substrate which may be only roughly 30 to 50 degrees Celsius as it exits the mold. Due to the thermal effusivity of polypropylene relative to the stainless steel of a mold roll, the small volume of small mass of polypropylene may substantially adopt the temperature of the stainless steel mold roll. In this case the material is effectively quenched. Even if the substrate is preheated to 150 degrees Celsius it will almost immediately quench to a mold temp (38° to 50° Celsius in this example) in the molding process with a portion of the substrate's initial thermal energy being transferred to the mold roll. Due to this cooling the material may need to be reheated prior to capping and bumping in the novel cap and post embodiment.

In one preferred embodiment a heated capping role is utilized which comprises a drum or cylindrical element heated to 200 to 250 degrees Celsius. The distal end of the protrusions from the cap and post formation from the ultrasonic formation process may ride along the circumference of this heated roll 90 to 160 to 180 to 270 or more degrees of wrap angle. Limiting the wrap angle may enable disengagement of the substrate during line stops. During this contact time conduction heating of the distal tips of the frustrations may soften and melt the protrusions. Finally, a backing roll which may be a solid steel roll may compress the protrusions against the heated roll.

Consistently formed protrusion heights may be achieved with a flat top. Downstream of this capping operation or preferentially close coupled, the substrate may be compressed against another roll such as a heated silicone rubber roll. As the distal tips of the hooks are heated and softened this may result in a softened head and around the heads as disclosed in prior art.

The surface speed of the capping roll (e.g. anvil roll 62) does not need to match the speed of the substrate 11 web or the surface velocity of the counter roll, such as the anvil roll. In certain capping operations, it may be possible to use an overspeed or underspeed of the capping roll to preferentially align the protrusions in a forward facing or backward facing machine direction. Such surface velocity of the capping roll may be +/−101%, 150%, 200%, 250%, 300% or more relative to said mold roll surface velocity.

All the techniques described for capping rolls may also be applied to deformable bumping rolls (e.g. deformable device 64), providing additional benefits. For instance, electric heaters such as cartridge heaters may be employed for heating capping rolls. Heater temperature can be feedback-controlled using sensing elements such as thermocouples or RTD resistance temperature detectors in conjunction with a programmable logic controller (PLC). Alternatively, heating can be achieved through a fluidic system utilizing a heated working fluid, such as heated glycol or oil. Such fluid may be circulated from an external heater through a rotary union into the rotating roll. The working fluid may then be distributed internally through internal porting to heat the entire roll or specific sections. For discrete heating sections, a cylindrical roll with a sleeve comprising thermally conductive and thermally insulating regions may be utilized. For example, a steel heated roll may incorporate aluminum or steel inserts that match the shape of a hook field for capping, allowing targeted heating. In cases where a heated effect is not desired, an insulating covering such as silicone rubber may be used to prevent damage to the nonwoven, film, laminate, or other substrate and intermediate materials, including the hooks.

As an alternate to traditional heat capping, bumping and/or capping may be performed with a rotary or blade style ultrasonic sonotrode. For example, a second rotary sonotrode add may be used to provide the capping/bumping operation. The ultrasonic sonotrode may be a blade style sonotrode or a rotary style sonotrode. Distal tips of the molded protrusions may be heated by various means, such as conductive, convective, or radiative heating, of the protruding cylindrical pins prior to capping. The ultrasonic capping operation may also provide a bonding function. For example, a roll with a profiled surface may provide capping of a discrete hook field to a known height in a first region. Said roll may also bond intermediate the hooks in a second region via a plurality of bonding projections on the roll intermediate the hook field capping regions.

A blade or rotary sonotrode may be employed for the capping/bumping operation(s). The ultrasonic capping operation may also provide a bonding function, enabling a capping of hook in a hook patch and bonding of a first hook containing substrate to a second substrate intermediate said hook patches. In this method, a hook substrate is formed, consisting of a first substrate for hook formation and a second substrate for bonding to a wider CD or longer MD substrate. Additionally, the capping operation may be used to selectively crush certain hooks, creating an intermediate effect in the hook region. For instance, products may have a region of no hooks adjacent to a cut edge, such as the primary fastening tab on a diaper, which can provide a soft aesthetic or be later cut to form a clean edge in subsequent operations.

This method may form a hook substrate may form which comprises of first substrate with integrally formed hook regions and a second substrate bonded to said first substrate. Said second substrate may be wider in a CD and/or longer in an MD product pitch than said first substrate.

The capping operation may be used to selectively deform or crush certain precursors protrusions to create regions with different protrusion properties. Some product embodiments may have a region of no hooks adjacent a cut edge in the finished product. For example, a primary fastening tab on a diaper back ear may have a first type of hooks in a first region of a hook field and a second type of hooks in a second region. The second type of hooks may comprise completely crushed hooks, shorter hooks, or hooks facing away from a cut edge proximate the edge of a fastening tape. The second hook region may provide a softer aesthetic at the cut edge and/or may provide a region which may be later cut in the subsequent operation to form a cut edge.

All the techniques described for capping rolls may also be applied to deformable bumping rolls, offering additional benefits. Capping and/or bumping rolls may be heated using electric heaters, such as cartridge heaters, with temperature control achieved through feedback control using sensing elements such as thermocouples or RTD resistance temperature detectors, in conjunction with a programmable logic controller (PLC). Alternatively, roll heating can be accomplished using a fluidic system, circulating a heated working fluid such as glycol, air, water, or oil from an external heater through a rotary union into the rotating roll, and then distributing said fluid internally to heat the entire roll or specific roll sections. For discrete heating sections, a roll with a sleeve comprising thermally conductive and insulating regions may be used. For instance, a steel heated roll may incorporate aluminum or steel inserts matching the shape of a hook field for capping, enabling targeted heating. Where heating is not desired, an insulating covering such as silicone rubber may be utilized to protect the nonwoven, film, laminate, or other substrate and intermediate materials, including the hooks. In some embodiments, the capping/bumping roll may be heated in at least some regions by induction heaters. Such inductive heating may be mounted statically around a portion of said roll, which may eliminate the slip rings, inductive couplings, and/or rotary unions of prior art. Non-contact thermocouples may be utilized to measure the roll temperature. In some embodiments, the distal tips of the protrusions may be heated directly, such as by convective heating such as via from hot air or radiative heating such as near infrared heaters.

Capping may be defined as deforming the distal region of a protrusion to create an enlarged cross-sectional area at the distal end. In prior art, such as U.S. Pat. No. 6,132,660, such capping is generally presented as forming a flat topped distal protuberance on an as-molded protrusion, but the protuberance does not require a flat top in the general case. Bumping may be defined as a secondary deformation after capping of the distal region of a protrusion. Bumping may be performed to round the distal surface and/or to create a reduced angle between a protrusion's post region and said protrusion's cap elements. A rounded distal region, such as a mushroom cap, may have aesthetic benefits such as a softer feel. A rounded distal region may result in improved penetration of a hook into a mating second substrate, such as a non-woven. A reduced cap to post angle may improve engagement of said hook with an individual fiber of a mating non-woven second substrate.

As described in U.S. Pat. No. 6,132,660, capping may be performed in prior art as simply running a plurality of as-molded protrusions through a heated nip. While adequate at low web speeds, this geometry provides very little in-nip residence time for heat transfer from the hot roller to the distal ends of the as-molded protrusions. U.S. Pat. No. 6,132,660 presents an improved arrangement, with the as-molded protrusion distal ends wrapping a roll for an increased angle, therefore increasing residence time. U.S. Pat. No. 6,132,660 utilizes a curved support structure to urge the precursor web against the heated roll.

The proposed novel solution of the method disclosed herein is to use a heated first roll to heat the distal tips of the precursor as-molded protrusion in a first step, and then compress said protrusions. This second compression step to a fixed dimension occurs between the heated first roll and a second roll. As shown in FIGS. 6A-6C, the substrate web may wrap the second roll.

The substrate may wrap the heated roll for an extended contact angle of about 160° or about 10° to about 270°. The wrap angle and roll temperature may be chosen to melt a certain distance of substrate from the distal tip, corresponding to the intended volume of the finished cap region. For the example 0.3 mm tall cylindrical protrusion of our example, the distal 0.166 mm may be raised near the melt temperature and the bottom 0.134 mm which defines the post may be at a significantly lower temperature. The actual temperature in the protrusion will be a gradient driven by conductive heat transfer through the material of the protrusion. For an example polypropylene with a melt temperature of about 162° C. and a softening temperature in the range of 110-140° C., a high temperature of about 200° C. to 250° C. may be utilized to reduce the required residence time. For other polymers a lower (e.g. polyethylene) or higher (e.g. PET) temperature range associated with said substrate's softening and melt temperatures may be chosen. A temperature of about 200° C. may be preferred to enable easier material options for insulator material. Residence time of a given hook against the hot anvil may be about 0.25 seconds or about 0.1 seconds to about 0.5 seconds. For a web speed of about 100 m/min (target range 30 m/min to about 300 m/min) this corresponds to a ˜200 mm diameter cylinder with a 160° contact angle.

As the heated roll may be higher temperature than a polymer, substrate may melt during line stops. The heated roll may be retractable away from the substrate. The substrate wrap angle may be chosen to be less than 180° or less than 170° or about 160° to facilitate such unloading. As the heated roller is a complex mechanism, in some embodiments a substrate web may be displaced to unload the substrate from the heated surface at line stops. For example, a first idler or a first and second idler pair may displace to reduce the substrate contact angle with the heated roll. Such displacement may be linear or rotary. Such displacement may be a rotation of an idler or idler pair about or proximate the axis of the heated roll. Such displacement may partially or fully remove the substrate web from the heated roll (FIG. 6C). The path length in run and bypass positions may be the same or nearly the same, to prevent the introduction of phase defects when switching between the two positions. A dancer element may facilitate such nominally equal path length. An air cylinder or spring loading mechanism may be utilized. An additional path length compensation idler or bar, which may only be operatively engaged in the bypass position, may be used to create a nominally equal path length. The lay-on idler may be proximate the heated roll in the run position and displaced radially from the heated troll in the bypass position. The bypass capability may facilitate wrap angles larger than 180°, which may be cumbersome to operate and/or execute. Wrap angles of 180° to about 350° may facilitate longer residence time, which may enable higher production line speeds. Wrap angles of 180° to about 350° may facilitate a smaller heated roll, which may reduce the cost and complexity of said heated roll.

Prior art hooks may be continuous in a machine direction and/or continuous in a cross machine (CD) direction. A key benefit of the unitary hook formation may be the capability to create discrete hook patches, or regions of a plurality of hooks with an appropriate mold roll. Such hook patch may be discrete or spaced apart in a machine direction. Such hook patches may be spaced apart in a cross machine direction. The heated roll may have discrete heat zones configured to be aligned in MD position, CD position, and/or machine phase with discrete as-molded precursor protrusion patches. The heated capping and/or bumping roll(s) may have a plurality of heated radial protrusions with a cylindrical surface, said protrusions corresponding to as-molded protrusion patches in the pre-cursor substrate. Intermediate such heated protrusions may be a thermal insulator material, such as a silicon rubber or PEEK (high temp engineering plastic). Such insulator may take from form of a cylindrical shell, which may have windowed cutouts around the heated radial protrusions of the heated roll. Such insulator shell may be segmented into segments, for example two hemispherical segments, to facilitate mounting to the heated roll. In some embodiments, the heated roll may be a heated cylindrical roll with a cylindrical sleeve. Said sleeve may comprise a conductive portion over with heated thermally conductive regions which match the precursor hook patches. Said sleeve may have insulator material where a heat effect is undesirable, such as intermediate hook patches in the precursor substrate. Said sleeve may be a cylindrical aluminum sleeve, with the outer surface relieved and/or machined to create islands of thermally conductive exposed surface. Said sleeve may have a silicon rubber or similar thermally insulating coating intermediate the thermally conductive regions. Said sleeve may be segmented, such as into two 180° split shells and mechanically fastener to the heated roll. Said sleeve may comprise a plurality of segments of a cylinder as removal inserts, said inserts being mechanically affixed and interchangeable in relevant positions on a heated roll. Such techniques may be utilized for size changeover.

### Further Definitions and Cross-References

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of creating projections in a substrate, comprising:
providing a first device comprising an outer surface, wherein the outer surface defines a plurality of recesses, wherein the recesses comprise a first portion proximate to the outer surface and having a first perimeter and a first depth, wherein the recesses comprise a second portion distal from the outer surface and having a second perimeter and a second depth, wherein the first perimeter is larger than the second perimeter, and wherein the first depth is less than the second depth, and wherein the first portion of the recesses substantially surrounds the second portion of the recesses;
providing a second device comprising a source of vibration energy;
forming a nip between the source of vibration energy and the outer surface;
conveying the substrate through the nip in a machine direction to create a plurality of precursor projections from a portion of the substrate, wherein the precursor projections have a first portion having a first perimeter and a first depth corresponding to the first perimeter and the first depth of the first portion of the recesses, wherein the precursor projections have a second portion having a second perimeter and a second depth corresponding to the second perimeter and the second depth of the second portion of the recesses, and wherein the first portion of the precursor projections substantially surrounds the second portion of the precursor projections;
wherein the plurality of precursor projections comprise proximal regions proximate to a plane of the substrate and distal regions distal from the plane of the substrate;
heating portions of the distal regions to a temperature at or above a melting temperature of the substrate; and
applying pressure to the portions of the distal regions to flatten the portions of the distal regions and increase a perimeter of the portion of the distal regions to form the projections.

2. The method of claim 1, wherein the heating the portions of the distal regions step comprises conveying the distal regions over a heated device.

3. The method of claim 1, wherein the applying pressure step comprises contacting the portions of the distal regions with an anvil.

4. The method of claim 1, comprising contacting the flattened distal regions with a deformable device to deform outer portions of the flattened distal regions toward the plane of the substrate.

5. The method of claim 4, wherein, after the contacting the flattened distal regions with the deformable device step, the projections comprise a mushroom shape.

6. The method of claim 1, wherein the heating the portions of the distal regions step is continuous.

7. The method of claim 1, wherein the heating the portions of the distal regions step is intermittent.

8. The method of claim 3, wherein the anvil is a metal anvil roll, and wherein the deformable device is a deformable roll.

9. The method of claim 1, wherein the substrate comprises a nonwoven or a film.

10. The method of claim 1, wherein the substrate comprises one or more layers.

11. The method of claim 1, comprising forming a shaped patch of the fully formed projections in the substrate.

12. The method of claim 11, comprising forming a plurality of the shaped patches intermittently in the substrate.

13. The method of claim 1, comprising imparting thermal energy to a portion of the substrate upstream of the nip to heat the portion of the substrate to a temperature below a melting temperature of the portion of the substrate.

14. The method of claim 1, wherein the projections are suitable for use as a portion of a touch fastener.

15. The method of claim 1, wherein the vibration energy is ultrasonic energy, and wherein the second device is a sonotrode.

16. The method of claim 1, wherein the first portions and the second portions of the projections are unitary.

17. The method of claim 1, wherein during the conveying step not more than 35% of the substrate is conveyed into the plurality of recesses to form the plurality of precursor projections.

18. The method of claim 1, wherein a ratio of a ported inline watershed area for each recess to an average watershed area for each recess is at least 50% or at least 70%.

19. A method of creating projections in a substrate, comprising:
providing a first device comprising an outer surface, wherein the outer surface defines a plurality of recesses, wherein the recesses comprise a first portion proximate to the outer surface and having a first perimeter and a first depth, wherein the recesses comprise a second portion distal from the outer surface and having a second perimeter and a second depth, wherein the first perimeter is larger than the second perimeter, and wherein the first depth is less than the second depth, and wherein the first portion of the recesses substantially surrounds the second portion of the recesses;
providing a second device comprising a source of heat or pressure;
forming a nip between the source of heat or pressure and the outer surface;
conveying the substrate through the nip in a machine direction to create a plurality of precursor projections from a portion of the substrate, wherein the precursor projections have a first portion having a first perimeter and a first depth corresponding to the first perimeter and the first depth of the first portion of the recesses, wherein the precursor projections have a second portion having a second perimeter and a second depth corresponding to the second perimeter and the second depth of the second portion of the recesses, and wherein the first portion of the precursor projections substantially surrounds the second portion of the precursor projections;
wherein the plurality of precursor projections comprise proximal regions proximate to a plane of the substrate and distal regions distal from the plane of the substrate;
heating portions of the distal regions to a temperature at or above a melting temperature of the substrate; and
applying pressure to the portions of the distal regions to flatten the portions of the distal regions and increase a perimeter of the portion of the distal regions to form the projections.

20. The method of claim 19, wherein the heating the portions of the distal regions step comprises conveying the distal regions over a heated device, comprising contacting the flattened distal regions with a deformable device to deform outer portions of the flattened distal regions toward the plane of the substrate.
